(19) United States
(12) Patent Application Publication
(10) Pub. No.: US 2024/0350497 A1
(43) Pub. Date: Oct. 24, 2024

(54) COMBINATION THERAPY FOR THE TREATMENT OF PAN-KRAS MUTATED CANCERS

(71) Applicant: Tiziana Life Sciences PLC, London (GB)

(72) Inventor: Thomas H. ADAMS, Rancho Santa Fe, CA (US)

(21) Appl. No.: 18/682,324

(22) PCT Filed: Aug. 8, 2022
(86) PCT No.: PCT/EP2022/072272

§ 371 Date: Feb. 8, 2024

Related U.S. Application Data
(60) Provisional application No. 63/231,130, filed on Aug. 9, 2021.

(51) Int. Cl. A61K 31/519; A61K 31/4745; A61K 31/513; A61K 31/555; A61K 31/65; A61K 31/7068; A61K 33/243; A61K 45/06; A61P 35/00

(52) U.S. Cl. CPC A61K 31/519; A61K 31/4745; A61K 31/513; A61K 31/555; A61K 31/65; A61K 31/7068; A61K 33/243; A61K 45/06; A61P 35/00

(57) ABSTRACT

This application relates to methods of treating and/or preventing cancer (e.g., non-small cell lung cancer, small cell lung carcinoma, colorectal cancer, pancreatic cancer, breast cancer, ovarian cancer, biliary cancer and melanoma in subjects in need thereof comprising administering to the patient a therapeutically effective amount of a CDK inhibitor (e.g., milciclib) in combination with a therapeutically effective amount of a DNA damaging agent.

### CROSS-REFERENCE TO RELATED APPLICATIONS

This application claims priority to and the benefit of U.S. Provisional Application No. 63/231,130, filed Aug. 9, 2021, the content of which is incorporated herein by reference in its entirety.

### TECHNICAL FIELD

This application relates generally to the treatment of KRAS mutated tumors and more particularly relates to the treatment of KRAS mutated tumors with a combination of a cyclin-dependent kinase (CDK) inhibitor and at least one DNA Damaging Agent.

### BACKGROUND

Milciclib, which may be referred herein to as Compound 1, or N,1,4,4-tetramethyl-8-((4-(4-methylpiperazin-1-yl)phenyl)amino)-4,5-dihydro-1H-pyrazolo[4,3-h]quinazoline-3-carboxamide, has the following structure:

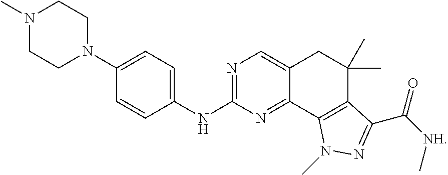 [SMILES: CNC(=O)c1nn(C)c2c1C(C)(C)Cc1cnc(Nc3ccc(N4CCN(C)CC4)cc3)nc1-2]

Milciclib is a small molecule inhibitor of multiple CDKs, including CDK1, CDK2, CDK4, CDK5, CDK7, and CDK9, and TRKs (TPKA and TRKC), has shown efficacy in several preclinical tumor models (Albanese C et al. (2010) Mol Cancer Ther 9:2243-2254). In a phase I study, oral treatment with milciclib was found to be well-tolerated and the drug showed promising clinical responses in patients with advanced solid malignancies such as in thymic carcinoma, pancreatic carcinoma and colon cancer (Weiss G J et al. (2013) Invest New Drugs 31:136-144.) The major toxicity profile consisted of tremors and gastrointestinal toxicity which was reversible upon treatment suspension. Results from this study recommended a RP2D of 150 mg/day.

Milciclib, exhibiting broad-spectrum inhibitory activities against CDKs, effectively retards proliferation of cancer cells. Therefore, it is reasonable to propose that anticancer activity of milciclib may be potentiated by an inhibitor of tyrosine kinase to produce synergistic anti-tumorigenic activity.

KRAS is the most frequently mutated oncogene in human carcinomas and mutations in KRAS can result in continuous cellular proliferation and cancer development. KRAS mutations are the most prevalent driver in lung cancer, making up 25% of adenocarcinomas. KRAS G12C is one of the most common driver mutations in NSCLC and there is a high unmet need, as well as poor outcomes associated in the second-line treatment of KRAS G12C driven NSCLC. In the U.S., about 13% of patients with NSCLC harbor the KRAS G12C mutation, and each year approximately 25,000 new patients in the U.S. are diagnosed with KRAS G12C-mutated NSCLC.

There is a need for novel therapies by using milciclib in combination with a second anticancer drug or agent for the treatment of KRAS mutated cancer. The present application addresses such a need.

### SUMMARY OF THE INVENTION

In one aspect, this application pertains to a method of treating or preventing cancer in a patient in need thereof, comprising administering to the patient a therapeutically effective amount of a CDK inhibitor, or a pharmaceutically acceptable salt, isomer, or tautomer thereof, in combination with a therapeutically effective amount of another anticancer drug.

In one aspect, the disclosure provides a method of treating a cancer in a subject in need thereof, the method comprising: a.) identifying a subject with a having a KRAS mutant tumor; and b.) administering milciclib to the subject. In one embodiments, the method further comprises administering a DNA damaging agent to the subject.

In some embodiments, the administration of milciclib and the DNA damaging agent is concurrent or sequential. In one embodiments, the DNA damaging agent is a poly adenosine diphosphate-ribose polymerase (PARP) inhibitor, a Topoisomerase I inhibitor, a Topoisomerase II inhibitor, an alkylating agent, an alkylating agent-steroid conjugate, an epoxide, a platin, an anthracenedione, an antimetabolite, an antifolate, a nucleic acid analog, a ribonucleic acid analog, a ribozyme, radiation, a vinca alkaloid, FOLFIRI, or a taxane. In some embodiments, the platin is cisplatin, oxaliplatin or carboplatin. In some embodiments, the antimetabolite is a gemcitabine, or a 5-fluorouracil. In some embodiments, the Topoisomerase I inhibitor is topotecan or irinotecan. In some embodiments, the Topoisomerase II inhibitor is anthracycline. In some embodiments, the alkylating agent is nitrogen mustard, a nitrourea, alkyl sulfonate a triazine, an aziridine or an ethylenimine.

In some embodiments, the KRAS mutant tumor has a one or more mutations anywhere on the KRAS gene. In some embodiments, the KRAS mutation occurs in codon 12, codon 13, or codon 61 of the KRAS gene. In some embodiments, the KRAS mutation is at least one of G12D, G12F, G12V, G12R, Q61H, G12C, G12S, G12L, Q61K, Q61R, A11T, G13C, G13P, G13D, and O51H.

In some embodiments, the cancer is selected from non-small cell lung cancer, colorectal cancer, pancreatic cancer, breast cancer, ovarian cancer, biliary cancer and melanoma.

In some embodiments, the subject has failed one or more previous treatment regimens. In some embodiments, the cancer is refractory to one or more prior administered chemotherapies. In some embodiments, the cancer is sensitized to the one or more prior administered therapies following administration of milciclib. In some embodiments, the cancer is gemcitabine-resistant prior to administering milciclib. In some embodiments, the cancer is sensitized to gemcitabine following administration of milciclib.

In some embodiments, the subject is a human.

In some embodiments, the milciclib is administered as a unit dose, wherein the unit dose is a therapeutically effective amount. In some embodiments, the unit dose is about 20 mg/kg, about 30 mg/kg, about 40 mg/kg, about 50 mg/kg, about 60 mg/kg, about 70 mg/kg, or about 80 mg/kg. In some embodiments, the unit dose is 20 mg per day, 25 mg per day, 30 mg per day, 35 mg per day, 40 mg per day, 45 mg per day, 50 mg per day, 55 mg per day, 60 mg per day, 65 mg per day, 70 mg per day, 75 mg per day, 80 mg per day, 85 mg per day, 90 mg per day, 95 mg per day, 100 mg per day, 105 mg per day, 110 mg per day, 115 mg per day, 120 mg per day, 125 mg per day, 130 mg per day, 135 mg per day, 140 mg per day, 145 mg per day, 150 mg per day, 155 mg per day, or 160 mg per day.

In some embodiments, the unit dose is administered orally. In some embodiments, the unit dose is administered once a day or twice a day. In some embodiments, the unit dose is administered for about 7 consecutive days, about 9 consecutive days, or about 15 consecutive days. In some embodiments, the unit dose is administered for a cycle of 7 days on followed by 7 days off, wherein the cycle is repeated for 4 weeks. In some embodiments, the unit dose is administered for a cycle of 4 days on followed by 3 days off, wherein the cycle is repeated for 4 weeks.

In some embodiments, the therapeutically effective amount of gemcitabine is 1000 mg/m² over 30 minutes once weekly for seven weeks, followed by one week of no administration, wherein the cycle is optionally repeated.

In some embodiments, the therapeutically effective amount of milciclib is 50, 75, 100, 125, or 150 mg once daily for four consecutive days, followed by non-administration for 3 consecutive days, wherein the cycle is optionally repeated.

In some embodiments, milciclib and the other anticancer drug are administered to the patient simultaneously. In some embodiments, milciclib and the other anticancer drug are administered in a single pharmaceutical formulation that further includes a pharmaceutically acceptable excipient. In some embodiments, milciclib and the DNA damaging agent are administered in temporal proximity.

In some embodiments, milciclib and the other anticancer drug are each administered in separate pharmaceutical formulations, wherein each formulation further includes a pharmaceutically acceptable excipient. In some embodiments, one or both of the pharmaceutical formulations is in a controlled release form. In some embodiments, the pharmaceutical formulation is in a controlled release form.

In some embodiments, milciclib and the other anticancer drug are administered to the subject sequentially. In some embodiments, administration of milciclib begins before administration of the other DNA damaging agent to the subject. In some embodiments, administration of milciclib begins after administration of the other anticancer to the subject.

In some embodiments, milciclib is administered in a single pharmaceutical formulation that further includes a pharmaceutically acceptable excipient. In some embodiments, the pharmaceutical formulation is formulated for oral administration. In some embodiments, the pharmaceutical formulation is in the form of a tablet, pill, or capsule.

In one aspect, the disclosure provides a pharmaceutical composition comprising milciclib or a pharmaceutically acceptable salt, isomer, or tautomer thereof, and another anticancer drug.

In one aspect, the disclosure provides a kit comprising: (a) a pharmaceutical composition comprising milciclib, or a pharmaceutically acceptable salt thereof; (b) a pharmaceutical composition comprising a poly adenosine diphosphate-ribose polymerase (PARP) inhibitor, a Topoisomerase I inhibitor, a Topoisomerase II inhibitor, an alkylating agent, an alkylating agent-steroid conjugate, an epoxide, a platin, an anthracenedione, an antimetabolite, an antifolate, a nucleic acid analog, a ribonucleic acid analog, a ribozyme, radiation, a vinca alkaloid, FOLFIRI, or a taxane, sorafenib, lenvatinib, regorafenib, sunitinib, nivolumab, gemcitabine, palbociclib, afatinib, alectinib, axitinib, bortezomib, bosutinib, cabozantinib, carfilzomib, ceritinib, cobimetinib, crizotinib, dabrafenib, erlotinib, gefitinib, ibrutinib, idelalisib, imatinib, ixazomib, lapatinib, nilotinib, nintedanib, niraparib, osimertinib, pazopanib, pegaptanib, ponatinib, rucaparib, ruxolitinib, sonidegib, tofacitinib, trametinib, vandetanib, vemurafenib, vismodegibor, or a pharmaceutically acceptable salt thereof, and (c) instructions for the use thereof in the treatment and/or prevention of cancer.

### BRIEF DESCRIPTION OF THE DRAWINGS

FIG. 1A is a diagram showing the steps of treating a cancer having a KRAS mutation in a subject with recurrent and/or metastatic cancer that has failed at least one line of therapy by co-administering milciclib and another chemotherapy.

FIG. 1B is a diagram showing a clinical trial design for determining efficacy of the co-administering milciclib and another chemotherapy.

FIG. 2 is a diagram showing regulation of the cell cycle by cyclin dependent kinases.

FIG. 3 is a diagram showing the distribution of specific KRAS mutations in non-small cell lung cancer.

FIG. 4A is a chart showing the frequency of mutations in pancreatic cancer. FIG. 4B is diagram showing the progression of mutations in pancreatic cancer. FIG. 4C is the distribution of KRAS mutations in pancreatic cancer.

FIG. 5 is a table showing the frequency of KRAS mutations in colorectal cancer.

FIG. 6A is a western blot showing the detection active cyclin dependent kinase 1 (pCDK1), total CDK1, and beta-actin loading control in isogenic cell lines containing various KRAS mutations. FIG. 6B is a western blot showing the amount of active cyclin dependent kinase 1 (pCDK1), total CDK1, and beta-actin loading control in a panel of non-isogenic cell lines. FIG. 6C is a western blot showing the amount of active cyclin dependent kinase 1 (pCDK1), total CDK1, and beta-actin loading control in a panel of colorectal non-isogenic cell lines. FIG. 6D is a series of charts illustrating the percentage of cells in G1, S, and G2/M phases of the cell cycle.

FIG. 7A and FIG. 7B are plots showing dose-response curves of colorectal non-isogenic cells containing either wild type (WT) or mutated KRAS in response to CDK inhibitors AT7519 (FIG. 7A) or dinaciclib (FIG. 7B).

FIG. 8A is a plot showing the change in tumor volume of KRAS mutant tumor xenografts in mice treated with vehicle control (DMSO) or the CDK inhibitor AZD5438.

FIG. 8B is a plot showing the percent survival of mice harboring KRAS mutant tumor xenografts following treatment with either vehicle control (DMSO) or the CDK inhibitor AZD5438. FIG. 8C is a chart showing the tumor weight in mice harboring KRAS mutant tumor xenografts following treatment with either vehicle control (DMSO) or the CDK inhibitor AZD5438. FIG. 8D is a series of images showing tumor shrinkage in mice harboring KRAS mutant tumor xenografts following treatment with the CDK inhibitor AZD5438.

FIG. 9A is a series of magnetic resonance imaging (MRI) images showing the change in tumor volume following treatment with either vehicle control or milciclib in a mouse model of pulmonary cancer. FIG. 9B is a plot showing the percent (%) tumor growth in a mouse model of pulmonary cancer treated with either vehicle control or milciclib.

FIG. 10A is a table showing the activity of milciclib (PHA-848125) in combination with 5-fluorouracil (5-FU) in animals harboring HCT-116 human colon carcinoma tumor xenografts. FIG. 10B is a table showing the activity of milciclib (PHA-848125) in combination with irinotecan in animals harboring HCT-116 human colon carcinoma tumor xenografts. FIG. 10C is a plot showing the change in tumor weight in animals harboring HCT-116 human colon carcinoma tumor xenografts following treatment with various doses of vehicle control, milciclib (PHA-125), irinotecan (CPT-11), or a combination of PHA-125 and CPT-11.

FIG. 11 is a table showing subjects having various cancers that show prolonged stable disease or partial response following treatment with milciclib and gemcitabine.

FIG. 12A and FIG. 12B are tables showing the activity of milciclib (PHA-848125) in combination with gemcitabine in animals harboring BX-PC3 human pancreatic carcinoma tumor xenografts. FIG. 12C is a plot showing the change in tumor weight in animals harboring BX-PC3 human pancreatic carcinoma tumor xenografts following treatment with various doses of vehicle control, gemcitabine, milciclib (PHA-125), or a combination of PHA-125 and gemcitabine.

FIG. 13A is a table showing the activity of milciclib (PHA-848125) in combination with topotecan in animals harboring N-592 human small cell lung carcinoma tumor xenografts. FIG. 13B is a plot showing the change in tumor weight in animals harboring N-592 human small cell lung carcinoma tumor xenografts following treatment with various doses of vehicle control, topotecan, milciclib (PHA-125), or a combination of PHA-125 and topotecan.

FIG. 14 is a table showing the tumor response in subjects having breast cancer following treatment with milciclib and gemcitabine.

FIG. 15A is a series of plots showing change in tumor volume in mice bearing MDA-MB-231 human breast cancer tumor xenografts following treatment with 40 mg/kg milciclib (PHA-848125) (n=8) or vehicle control (5% glucose solution) (n=8) via oral gavage twice daily on a 5-days-on/2-days-off schedule for four weeks. FIG. 15B is a series of images and a chart showing immunohistochemistry staining of Ki67 and mitotic nucleus counts, and corresponding quantification in tumor tissue collected 48 hours after the last treatment with milciclib (PHA-848125) or vehicle control for 3 weeks.

FIG. 16A is a table showing that effective doses of milciclib (PHA-848125) in combination with various chemotherapies (paclitaxel, doxorubicin, 5-fluorouracil, cisplatin, and cyclophosphamide) suppresses the growth of MDA-MB-231 human breast cancer cells in vitro. FIG. 16B is a chart showing the change in tumor volume in mice harboring tumor xenoplants treated with vehicle control, PHA848125 (40 mg/kg twice a day for 5 days for two weeks by gavage), cisplatin alone (5 mg/kg three times per week for two weeks by intraperitoneal injection) or in combination. FIG. 16C is a series of images showing tumor burden in mice harboring tumor xenoplants following treatment with vehicle control, milciclib (PHA848125) (40 mg/kg twice a day for 5 days for two weeks by gavage), cisplatin alone (5 mg/kg three times per week for two weeks by intraperitoneal injection) or in combination (black triangles indicate presence of tumor).

FIG. 17 is a plot showing change in tumor weight in mice bearing A2780 human ovarian tumor xenografts following oral administration of milciclib at various concentrations. TGI=tumor growth inhibition.

FIG. 18 is a table showing the in vivo efficacy of milciclib on a panel of mice bearing tumor xenoplants, including ovarian cancer, prostate cancer, acute myeloid leukemia, pancreatic cancer, non-small cell lung cancer, breast cancer, pancreatic cancer, melanoma, and colon cancer.

FIG. 19 is a series of diagrams showing the direct cytotoxic effects of poly ADP-ribose polymerases.

### DETAILED DESCRIPTION OF THE INVENTION

The present disclosure is based upon the surprising discovery of synergistic effects for milciclib in combination with a DNA damaging agent, in the reduction of tumor formation and progression in cancer cells. Additionally, the combination treatment of milciclib with the DNA damaging agent, gemcitabine, shows not only significant disease stabilization, but also evidence that milciclib has the astounding ability to reverse gemcitabine-resistance in refractory solid tumors. Furthermore, it has been shown that milciclib can selectively target KRAS-mutated cancers. Across various studies, milciclib has shown inhibitory effects against multiple cell lines with mutationally active G12D (non-small cell lung cancer), G13D (colorectal cancer), G12V (pancreatic cancer), and G12C (pancreatic cancer).

Accordingly, the present disclosure provides methods of treating subjects having cancer by identifying subjects with harboring multiple KRAS mutations Combination therapy of milciclib and cytotoxic agents, e.g., DNA damaging agents to target pan-KRAS mutated carcinoma in patients who have previously failed first line treatment has the potential to greatly improve the efficacy and outcome of current treatments.

RAS activity regulates a complex signaling network, including the RAF-MEK-ERK cascade, the phosphatidylinositol 3-kinase pathway and the effector family of exchange factors for the RAL small GTPases. KRAS (Kirsten Rat Sarcoma virus), a member of the RAS family, is a key regulator of signaling pathways that are responsible for cell proliferation, differentiation, and survival. KRAS encodes small G proteins with intrinsic GTPase activity. KRAS is the most frequently mutated oncogene in human carcinomas and mutations in KRAS can result in continuous cellular proliferation and cancer development. KRAS mutations are the most prevalent driver in lung cancer, making up 25% of adenocarcinomas.

KRAS oncogene is frequently mutated in human tumors and activating mutations in KRAS occur in 20-30% of NSCLC. KRAS mutations occur mainly in codon 12, 13 or 61. The most common types of KRAS mutations are G12C (42%), G12V (21%), and G12D (17%).

The KRAS G12C is a single point mutation with a glycine-to-cysteine substitution at codon 12. This substitution favors the activated state of KRAS, amplifying signaling pathways that lead to oncogenesis.

A pan-KRAS mutated cancer treatment could clinically improve the outcomes for many patients. In non-small cell lung cancer (NSCLC) up to 30% of all NSCLC patients possess a KRAS mutation. Additionally KRAS is mutationally active in 94% of pancreatic ductal adenocarcinoma (PDAC). The most common mutations are G12D (41%), G12V (34%), and G12R (16%)3. The G12C mutation is rare in PDAC, with only 1% of all KRAS mutations.

### Methods of Treatment

Milciclib has significant antitumor activity in various human xenografts and carcinogen-induced tumors. It is shown to be potent dual inhibitor of CDK2 and TrkA, which are essential for cell cycle proliferation. In vivo activity of milciclib in human xenograft tumor models in nudemice showed the compound to be effective and well tolerated.

Without wishing to be bound by theory, it is believed that for the treatment of cancers with KRAS mutations, a combination treatment of milciclib in addition to a DNA damaging agent would be favorable. These agents are hypothesized to induce apoptosis to subsequently eliminate cancer cells from the body. The activity of milciclib has been tested in combination with the following DNA damaging agents: gemcitabine, 5FU, irinotecan and topotecan for pancreatic carcinoma, colon carcinoma, colon carcinoma, and small cell lung carcinoma, respectively. After screening milciclib for in vivo activity in combination with various anticancer agents in xenograft models, the activity of combination treatment was superior to that of each drug alone. It can be safely combined with other anticancer drugs to improve their efficacy.

The present application provides methods of treating cancer, comprising administering to a subject having a KRAS mutated cancer a therapeutically effective amount of milciclib, or a pharmaceutically acceptable salt thereof, with one or more pharmaceutically acceptable carriers or excipients, in combination with a DNA damaging agent, with one or more pharmaceutically acceptable carriers or excipients, wherein the cancer is treated. In one embodiment, the anticancer drug is any compound disclosed herein other than milciclib.

In some embodiments, the cancer is any cancer that has one or more mutation in the KRAS gene. For example, the cancer may have at least 2, 3, 4, or more mutations in the KRAS gene. KRAS mutations include for example mutation resulting in the following amino acid substitutions: G12D, G12F, G12V, G12R, Q61H, G12C, G12S, G12L, Q61K, Q61R, A11T, G13C, G13P, G13D, and O51H.

In some embodiments, the cancer is a solid tumor (or tumors), or a refractory solid tumor (or tumors).

The cancer may be, for example, non-small cell lung cancer, colorectal cancer, pancreatic cancer, breast cancer, ovarian cancer, biliary cancer and melanoma.

In some embodiments, the method of treating cancer includes a reduction in tumor size. Alternatively, or in addition, the cancer may be a metastatic cancer and this method of treatment includes inhibition of metastatic cancer cell invasion.

The DNA damaging agent can be poly-adenosine diphosphate-ribose polymerase (PARP) inhibitor, a Topoisomerase I inhibitor, a Topoisomerase II inhibitor, an alkylating agent, an alkylating agent-steroid conjugate, an epoxide, a platin drug, an anthracycline, an anthracenedione, an antimetabolite, an antifolate, a nucleic acid analog, a ribonucleic acid analog, a ribozyme, radiation, a vinca alkaloid, FOLFIRI, or a taxane.

In one embodiment, the DNA damaging agent is an anti-metabolite or a nucleoside analog. Exemplary anti-metabolites or nucleoside analogs include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS). Preferably, the anti-metabolite is gemcitabine (Gemzar) or 5-fluorouracil.

In another embodiment, the DNA damaging agent is a Topoisomerase I inhibitor (TOPO I) inhibitor). TOPO I inhibitors include camptothecins and non-camptothecins podophyllotoxins. Exemplary camptothecins include, topotecan (TPT), irinotecan, and belotecan

Non-camptothecins, include for example indolocarbazoles, topovale (ARC-111), indotecan (LMP-400) and indimitecan (LMP-776),

In another embodiment, the DNA damaging agent is a Topoisomerase II inhibitor (TOPO II inhibitor). TOPO II inhibitors include quinolones, fluoroquinolones coumarin, and simocyclinones. Exemplary TOPO II inhibitors include nalidixic acid, cinoxacin, norfloxacin, ciprofloxacin, levofloxacin, sparfloxacin, moxifloxacin, doxorubicin, daunorubicin (doxorubicin precursor), Epirubicin (a doxorubicin stereoisomer), Idarubicin (a daunorubicin derivative), etoposide, teniposide, dexrazoxane, novobiocin, merbarone, anthracycline and aclarubicin.

In other embodiments, the DNA damaging agent is a platin drug. Exemplary platin drug include is cisplatin, oxaliplatin and carboplatin.

In some embodiments the, the DNA damaging agent is an is nitrogen mustard (e.g., bendamustine, chlorambucil, cyclophosphamide, ifosfamide, mechlorethamine, and melphalan), a nitrourea (e.g., carmustine, lomustine, and streptozocin), alkyl sulfonate, (e.g., busulfan) a triazine (e.g., dacarbazine and temozolomide), an aziridine or an ethylenimine (e.g., altretamine and thiotepa).

Milciclib or a pharmaceutically acceptable salt thereof, and/or the other anticancer drug, can be incorporated into pharmaceutical compositions suitable for administration. Such compositions typically comprise the compound (i.e. including the active compound), and a pharmaceutically acceptable excipient or carrier. As used herein, “pharmaceutically acceptable excipient” or “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field. Preferred examples of such carriers or diluents include, but are not limited to, water, saline, ringer's solutions, dextrose solution, and 5% human serum albumin.

Pharmaceutically acceptable carriers include solid carriers such as lactose, terra alba, sucrose, talc, gelatin, agar, pectin, acacia, magnesium stearate, stearic acid and the like. Exemplary liquid carriers include syrup, peanut oil, olive oil, water and the like. Similarly, the carrier or diluent may include time-delay material known in the art, such as glyceryl monostearate or glyceryl distearate, alone or with a wax, ethylcellulose, hydroxypropylmethylcellulose, methylmethacrylate or the like. Other fillers, excipients, flavorants, and other additives such as are known in the art may also be included in a pharmaceutical composition according to this application. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In one aspect, milciclib, or a pharmaceutically acceptable salt thereof, and/or the DNA damaging agent, is administered in a suitable dosage form prepared by combining a therapeutically effective amount (e.g., an efficacious level sufficient to achieve the desired therapeutic effect through inhibition of tumor growth, killing of tumor cells, etc.) of milciclib, or a pharmaceutically acceptable salt thereof (as an active ingredient) and/or the DNA damaging agent, with standard pharmaceutical carriers or diluents according to conventional procedures (i.e., by producing a pharmaceutical composition of the application). These procedures may involve mixing, granulating, and compressing or dissolving the ingredients as appropriate to attain the desired preparation.

As used herein, “treating” describes the management and care of a subject for the purpose of combating a disease, condition, or disorder and includes decreasing or alleviating the symptoms or complications, or eliminating the disease, condition or disorder.

As used herein, “preventing” describes stopping the onset of the symptoms or complications of the disease, condition or disorder.

In one aspect, treating cancer results in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. In a preferred aspect, size of a tumor may be measured as a diameter of the tumor.

In another aspect, treating cancer results in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

In another aspect, treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. In a preferred aspect, number of tumors may be measured by counting tumors visible to the naked eye or at a specified magnification. In a preferred aspect, the specified magnification is 2×, 3×, 4×, 5×, 10×, or 50×.

In another aspect, treating cancer results in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. In a preferred aspect, the number of metastatic lesions may be measured by counting metastatic lesions visible to the naked eye or at a specified magnification. In a preferred aspect, the specified magnification is 2×, 3×, 4×, 5×, 10×, or 50×.

In another aspect, treating cancer results in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active compound. In another preferred aspect, an increase in average survival time of a population may also be measured, for example, by calculating for a population the average length of survival following completion of a first round of treatment with an active compound.

In another aspect, treating cancer results in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active compound. In another preferred aspect, an increase in average survival time of a population may also be measured, for example, by calculating for a population the average length of survival following completion of a first round of treatment with an active compound.

In another aspect, treating cancer results in increase in average survival time of a population of treated subjects in comparison to a population receiving monotherapy with a drug that is not milciclib, or a pharmaceutically acceptable salt, prodrug, metabolite, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active compound. In another preferred aspect, an increase in average survival time of a population may also be measured, for example, by calculating for a population the average length of survival following completion of a first round of treatment with an active compound.

In another aspect, treating cancer results in a decrease in the mortality rate of a population of treated subjects in comparison to a population receiving carrier alone. In another aspect, treating cancer results in a decrease in the mortality rate of a population of treated subjects in comparison to an untreated population. In a further aspect, treating cancer results a decrease in the mortality rate of a population of treated subjects in comparison to a population receiving monotherapy with a drug that is not milciclib, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. In a preferred aspect, a decrease in the mortality rate of a population of treated subjects may be measured by any reproducible means. In another preferred aspect, a decrease in the mortality rate of a population may be measured, for example, by calculating for a population the average number of disease-related deaths per unit time following initiation of treatment with an active compound. In another preferred aspect, a decrease in the mortality rate of a population may also be measured, for example, by calculating for a population the average number of disease-related deaths per unit time following completion of a first round of treatment with an active compound.

In another aspect, treating cancer results in a decrease in tumor growth rate. Preferably, after treatment, tumor growth rate is reduced by at least 5% relative to the tumor growth rate prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. In a preferred aspect, tumor growth rate is measured according to a change in tumor diameter per unit time.

In another aspect, treating cancer results in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. In a preferred aspect, tumor regrowth is measured, for example, by measuring an increase in the diameter of a tumor after a prior tumor shrinkage that followed treatment. In another preferred aspect, a decrease in tumor regrowth is indicated by failure of tumors to reoccur after treatment has stopped.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3d ed.), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18th edition (1990). These texts can, of course, also be referred to in making or using an aspect of the application.

The term “controlled release” or “controlled release form” refers to a drug-containing formulation or fraction thereof in which release of the drug is not immediate, i.e., with a “controlled release” formulation, administration does not result in immediate release of the drug into an absorption pool. The term is used interchangeably with “non-immediate release” as defined in Remington: The Science and Practice of Pharmacy, Nineteenth Ed. (Easton, PA: Mack Publishing Company, 1995). In general, the term “controlled release” as used herein includes sustained release and delayed release formulations.

The term “sustained release” (synonymous with “extended release”) is used in its conventional sense to refer to a drug formulation that provides for gradual release of a drug over an extended period of time, and that preferably, although not necessarily, results in substantially constant blood levels of a drug over an extended time period. The term “delayed release” is also used in its conventional sense, to refer to a drug formulation which, following administration to a patient, provides a measurable time delay before drug is released from the formulation into the patient's body.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” derivative or analog, refers to a derivative or analog having the same type of pharmacological activity as the parent compound and approximately equivalent in degree.

Administration of the active agents may be carried out using any appropriate mode of administration. Thus, administration can be, for example oral or parenteral, although oral administration is preferred.

Depending on the intended mode of administration, the pharmaceutical formulation may be a solid, semi-solid or liquid, such as, for example, a tablet, a capsule, a caplet, a liquid, a suspension, an emulsion, a suppository, granules, pellets, beads, a powder, or the like, preferably in unit dosage form suitable for single administration of a precise dosage. Suitable pharmaceutical formulations and dosage forms may be prepared using conventional methods known to those in the field of pharmaceutical formulation and described in the pertinent texts and literature, e.g., in Remington: The Science and Practice of Pharmacy (Easton, PA: Mack Publishing Co., 1995). Oral administration and therefore oral dosage forms are generally preferred, and include tablets, capsules, caplets, solutions, suspensions and syrups, and may also comprise a plurality of granules, beads, powders, or pellets that may or may not be encapsulated. Preferred oral dosage forms are capsules and tablets.

As noted above, it is especially advantageous to formulate compositions of the invention in unit dosage form for ease of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of each active agent, such that the unit dosage in each tablet or capsule is approximately 50% of the therapeutically effective amount.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like.

Capsules are also preferred oral dosage forms, in which case the active agent-containing composition may be encapsulated in the form of a liquid or solid (the latter including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited earlier herein, which describes materials and methods for preparing encapsulated pharmaceuticals.

Generally, as will be appreciated by those of ordinary skill in the art, sustained release dosage forms are formulated by dispersing the active agents within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Sustained release dosage forms herein may be composed of the acrylate and methacrylate copolymers available under the tradename “Eudragit” from Rohm Pharma (Germany). The Eudragit series E, L, S, RL, RS, and NE copolymers are available as solubilized in organic solvent, in an aqueous dispersion, or as a dry powder. Preferred acrylate polymers are copolymers of methacrylic acid and methyl methacrylate, such as the Eudragit L and Eudragit S series polymers. In one embodiment, any of the pharmaceutical formulations may be formulated for sustained release, i.e., in a sustained release dosage form.

Preparations according to this invention for parenteral administration include sterile aqueous and non-aqueous solutions, suspensions, and emulsions. Injectable aqueous solutions contain the active agent in water-soluble form. Examples of non-aqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

Each of the active agents may in addition be administered through the skin using conventional transdermal drug delivery systems, wherein the active agent or agents are contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer.

In addition to the formulations described previously, the active agents may be formulated in a depot preparation for controlled release of the active agents, preferably sustained release over an extended time period. These sustained release dosage forms are generally administered by implantation (e.g., subcutaneously or intramuscularly or by intramuscular injection).

A “daily dose” of a particular material refers the amount of the material administered in a day. A daily dose can be administered as a single dose or as multiple doses. When a daily dose is administered as multiple doses, the daily dose is the sum of the amount of material administered in all of the multiple doses that are administered over the course of one day. For example, a daily dose of 12 mg can be administered in a single 12 mg dose once per day, in 6 mg doses administered twice per day, in 4 mg doses administered three times per day, in 2 mg doses administered six times per day, etc. The multiple doses can be the same or different doses of the material, unless otherwise specified. When a daily dose is administered as multiple doses, the multiple doses can be administered by the same or different route of administration, unless otherwise specified. Thus, a daily dose of 12 mg can include, for example, a 10 mg intramuscular dose and a 2 mg oral dose administered over the course of one day.

Administration of one compound “with” a second compound, as used herein, includes but is not limited to cases where the two compounds are administered simultaneously or substantially simultaneously. For example, administration of a first compound with a second compound can include administering the first compound in the morning and administering the second compound in the evening, as well as administering the first and second compounds in the same dosage form or in two different dosage forms that at the same or nearly the same time.

In combining the active agents disclosed herein, i.e., milciclib with another anticancer drug or agent disclosed herein, milciclib will generally reduce the quantity of the second drug or agent needed to achieve a therapeutic effect when administered as a monotherapy, and, conversely, the other anticancer drug or agent will generally reduce the quantity of milciclib required.

As the method of the application involves combination therapy, the active agents may be administered separately, at the same or at different times of day, or they may be administered in a single pharmaceutical formulation.

In some embodiments, “temporal proximity” means that administration of the other anticancer drug occurs within a time period before or after the administration of the CDK inhibitor (e.g., milciclib), such that the therapeutic effect of the other kinase inhibitor drug overlaps with the therapeutic effect of the CDK inhibitor (e.g., milciclib). In some embodiments, the therapeutic effect of the other kinase inhibitor drug completely overlaps with the therapeutic effect of the CDK inhibitor (e.g., milciclib). In some embodiments, “temporal proximity” means that administration of the other kinase inhibitor drug occurs within a time period before or after the administration of the CDK inhibitor (e.g., milciclib), such that there is a synergistic effect between the other kinase inhibitor drug and the CDK inhibitor.

“Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

### Pharmaceutical Compositions and Formulations

A pharmaceutical composition of the application is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the application can be administered to a subject in many of the well-known methods currently used for chemotherapeutic treatment. For example, for treatment of cancers, a compound of the application may be injected directly into tumors, injected into the blood stream or body cavities or taken orally or applied through the skin with patches. The dose chosen should be sufficient to constitute effective treatment but not so high as to cause unacceptable side effects. The state of the disease condition and the health of the patient should preferably be closely monitored during and for a reasonable period after treatment.

The term “therapeutically effective amount,” as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

The therapeutically effective amount of milciclib is 1-500 mg administered one or more times over a day for up to 30 or more days, followed by 1 or more days of non-administration of milciclib. This type of treatment schedule, i.e., administration of milciclib on consecutive days followed by non-administration of milciclib on consecutive days may be referred to as a treatment cycle. A treatment cycle may be repeated as many times as necessary to achieve the intended affect.

In one embodiment, the therapeutically effective amount of milciclib is 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 mg once or twice daily for one, two, three, four, five, six, seven, eight, nine, ten, eleven, twelve, thirteen, fourteen, or fifteen consecutive days, followed by non-administration for one, two, three, four, five, six, or seven consecutive days, wherein the cycle is optionally repeated 1, 2, or 3 times.

In one embodiment, the therapeutically effective amount of milciclib is 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mg once or twice daily for one, two, three, four, five, six, seven, eight, nine, or ten consecutive days, followed by non-administration for one, two, three, four, five, six, or seven consecutive days, wherein the cycle is optionally repeated 1, 2, or 3 times.

In one embodiment, the therapeutically effective amount of milciclib is 40, 45, 50, 55, 60, 65, 70, 75, 80, 85, 90, 95, 100, 105, 110, 115, 120, 125, 130, 135, 140, 145, or 150 mg once or twice daily for one, two, three, four, five, six, or seven consecutive days, followed by non-administration for one, two, three, four, five, six, or seven consecutive days, wherein the cycle is optionally repeated 1, 2, or 3 times.

In one embodiment, the therapeutically effective amount of milciclib is 75, 80, 85, 90, 95, 100, 105, 110, 115, 120, or 125 mg once daily for four consecutive days, followed by non-administration for three consecutive days, wherein the cycle is optionally repeated 1, 2, or 3 times.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present application may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one aspect, the active compounds are prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the application are dictated by and directly dependent on the unique characteristics of the active compound and the particular therapeutic effect to be achieved.

In therapeutic applications, the dosages of the pharmaceutical compositions used in accordance with the application vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 3000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can include co-formulations of milciclib and any of the compounds described herein.

The pharmaceutical compositions can be included in a container, pack, or dispenser together with instructions for administration.

It must be noted that, as used in this specification and the appended claims, the singular forms “a,” “an” and “the” include plural referents unless the context clearly dictates otherwise. Thus, for example, “a CDK inhibitor” refers not only to a single inhibitor but also to a combination of two or more different inhibitors, “a dosage form” refers to a combination of dosage forms as well as to a single dosage form, and the like.

Unless defined otherwise, all technical and scientific terms used herein have the meaning commonly understood by one of ordinary skill in the art to which the invention pertains. Specific terminology of particular importance to the description of the present invention is defined below.

As used herein, the term “patient” or “individual” or “subject” refers to any person or mammalian subject for whom or which therapy is desired, and generally refers to the recipient of the therapy to be practiced according to the invention. The terms “subject” and “patient” are used interchangeably herein.

As used herein, the term “CDK inhibitor” refers to a compound that inhibits the enzyme in humans referred to as cyclin-dependent kinase. Examples include, without limitation, milciclib, palbociclib, dinaciclib, P276-00, roniciclib, ribociclib, P1446A-05, AT7519M, SNS-032, SCH 727965, AG-024322, hygrolidin, fascaplysin, abemaciclib, arcyriaflavin A, CINK4, AM-5992, CDK4 Inhibitor (CAS #546102-60-7), CDK4 Inhibitor III (CAS #265312-55-8), Cdk4/6 Inhibitor IV (CAS #359886-84-3), MM-D37K, NSC 625987, ON-123300, or any pharmaceutically acceptable salt thereof. (See Law, M. E. et al. “Cyclin-Dependent Kinase Inhibitors as Anticancer Therapeutics” Mol. Pharmacol. 88:846-852 (2015), which is incorporated by reference herein in its entirety). In one embodiment, the CDK inhibitor is milciclib.

When referring to an active agent, applicant intends the term “active agent” to encompass not only the specified molecular entity but also its pharmaceutically acceptable, pharmacologically active analogs, including, but not limited to, salts, esters, amides, prodrugs, conjugates, active metabolites, crystalline forms (including polymorphs), enantiomers, and other such derivatives, analogs, and related compounds.

The terms “treating” and “treatment” include the following actions: (i) preventing a particular disease or disorder from occurring in a subject who may be predisposed to the disease or disorder but has not yet been diagnosed as having it; (ii) inhibiting the disease, i.e., arresting its development; or (iii) relieving the disease by reducing or eliminating symptoms and/or by causing regression of the disease.

The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of milciclib, such that the unit dosage in each tablet or capsule is approximately 50% of the therapeutically effective amount.

By the terms “effective amount” and “therapeutically effective amount” of a compound is meant a nontoxic but sufficient amount of an active agent to provide the desired effect, i.e., treatment of cancer.

As used herein, a “subject in need thereof” is a subject having cancer, or a subject having an increased risk of developing cancer relative to the population at large.

The term “cancer” includes solid tumors, as well as, hematologic tumors and/or malignancies. A “cancer cell” or “cancerous cell” is a cell manifesting a cell proliferative disorder that is a cancer. Any reproducible means of measurement may be used to identify cancer cells. Cancer cells can be identified by histological typing or grading of a tissue sample (e.g., a biopsy sample). Cancer cells can be identified through the use of appropriate molecular markers.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular carcinoma, hepatocellular (liver) cancer, Hodgkin lymphoma, hypopharyngeal cancer, intraocular melanoma, ocular cancer, islet cell tumors (endocrine pancreas), Kaposi's sarcoma, kidney cancer (renal cell carcinoma), renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstrom macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, thyroid carcinoma, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

### OTHER EMBODIMENTS

It is to be understood that while the invention has been described in conjunction with the preferred specific embodiments thereof, that the foregoing description is intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains.

### EXAMPLES

### Example 1: Co-Administration of Milciclib and Another Chemotherapy Agent in Subjects Having a Cancer

Milciclib is a potent dual inhibitor of CDK2 and TrKA and the combination of inhibition both decreases cell proliferation and invasiveness of cells. It is not limited to inhibiting one cyclin-dependent kinase, as it also inhibits CDK1, CDK4, CDK5, and CDK7 as well. Study data proves milciclib can selectively target KRAS mutant tumors in transgenic mouse models. Efficacy studies have shown tumor inhibition by milciclib treatment in multiple cancers, including ovarian cancer and melanoma. Data shows synergistic effects for milciclib in combination with cisplatin with reduction of tumor formation and progression in TNBC cells.

Strong clinical data from a Phase I dose-escalation study for the combination treatment of milciclib plus the DNA damaging agent, gemcitabine, shows not only significant disease stabilization, but also evidence that milciclib has the astounding ability to reverse gemcitabine resistance in refractory solid tumors. Data shows that the treatment of milciclib is synergized with a DNA damaging agent, such as gemcitabine, 5-FU, topotecan, irinotecan or PARP inhibitors. Screening for KRAS synthetic lethal interaction with the CDK1 in tumors harboring multiple KRAS mutations is a more effective approach, compared to targeting only one KRAS mutation such as G12C in NCSLC. As a second line treatment in patients who have previously failed first line therapy, milciclib, in combination with a DNA damaging agent, can target pan-KRAS mutated cancer, such as lung, pancreatic, and colorectal carcinomas, to significantly improve efficacy and positively impact patients outcomes.

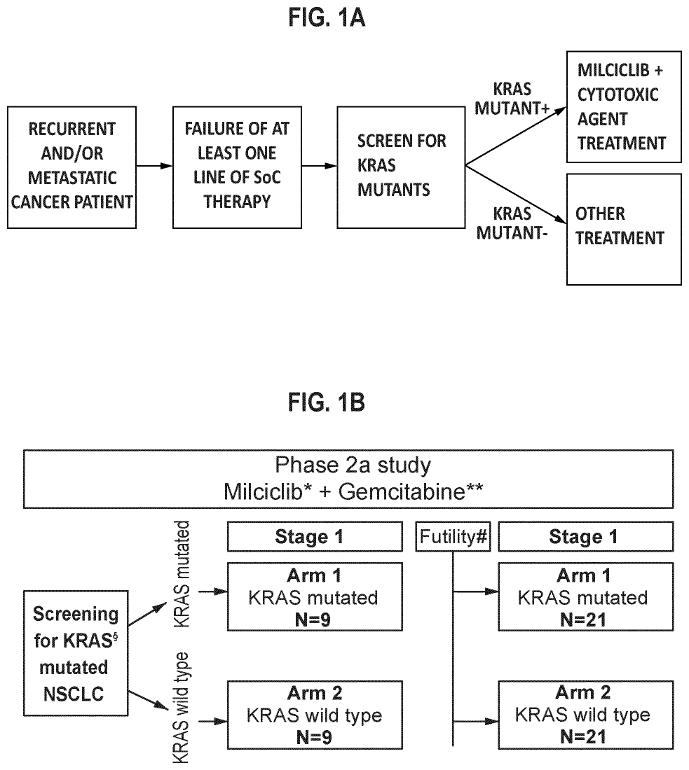

To address the possibility that KRAS drives a series of effects that induce CDK1 synthetic lethality and that milciclib is synergized with cytotoxic agents to increase tumor growth control, a Phase IIa clinical trial for the combination therapy of milciclib and gemcitabine in pan-KRAS mutated NSCLC subjects will be performed. The clinical trial will be a multicenter, multinational, prospective, non-randomized, open-label, parallel-arm Phase IIa clinical trial in locally advanced non-resectable recurrent and/or metastatic NSCLC after failure of at least one line of SoC therapy (FIG. A1 and FIG. 1B). All subjects will be screened for KRAS mutation and will be divided into the following two arms (up to 30 subjects in each arm): (1) KRAS-mutation positive for G12V, G12D, G12S, and G13D and (2) KRAS-mutation wildtype. The recommended dose will be 150 mg/day for milciclib and 1000 mg/m2/day of gemcitabine. Milciclib will be administered orally once daily for 7 days on/7 days off in a 4-week cycle and gemcitabine will be administered intravenously on days 1, 8, and 15. Tumor responses will initially be measured and recorded every 8 weeks (or after every other cycle in case of delay) using the RECIST (Response Evaluation Criteria in Solid Tumours) classification over the first 24 weeks, and then after every 3 cycles. Safety data will be collected on a continuous observation basis from the first dose of study drug through 30 days after the last dose or before initiation of a new anti-neoplastic treatment, whichever occurs first. Blood samplings for PK, and for exploratory endpoints will be performed (not mandatory). The subject's plasma will be measured for KRAS mutation load by ct-DNA analysis at appropriate time intervals.

### Example 2: Milciclib is an Inhibitor of Cyclin Dependent Kinases and TrkA

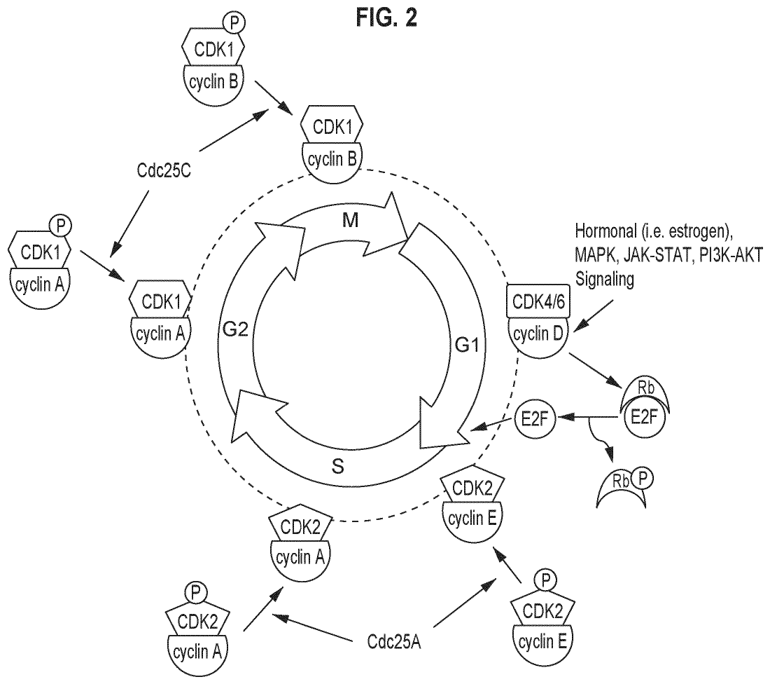

CDK2, CDK4, and CDK1 play a crucial role in progression of the cell cycle. Specifically, milciclib inhibits CDK2, which is the master regulator of S phase entry for cell cycle progression, required for proper DNA repair, and stimulation of the DNA Damage Response (DDR). CDK2 is responsible for the facilitation the phosphorylation of the Retinoblastoma (Rb), which releases E2F, and then allows for S-phase entry (FIG. 2).

The TrkA enzyme is activated following binding to neurotrophins, such as nerve growth factor (NGF). The TrkA/NGF signaling pathway promotes the survival, proliferation, and invasiveness of cells. Inhibition of both CDK2 and TrkA blocks cancer cell proliferation and reduces cancer growth. Milciclib treatment shuts down multiple key cellular roles to stop cancer growth by preventing cancer cells from entering cell replication and interfering with DNA repair, including activation of the DDR.

### Example 3: KRAS Mutations Contribute to a Variety of Cancers

RAS activity regulates a complex signaling network, including the RAF-MEK-ERK cascade, the phosphatidylinositol 3-kinase pathway and the effector family of exchange factors for the RAL small GTPases. KRAS (Kirsten Rat Sarcoma virus), a member of the RAS family, is a key regulator of signaling pathways that are responsible for cell proliferation, differentiation, and survival. KRAS encodes small G proteins with intrinsic GTPase activity. KRAS is the most frequently mutated oncogene in human carcinomas and mutations in KRAS can result in continuous cellular proliferation and cancer development. KRAS mutations are the most prevalent driver in lung cancer, making up 25% of adenocarcinomas.

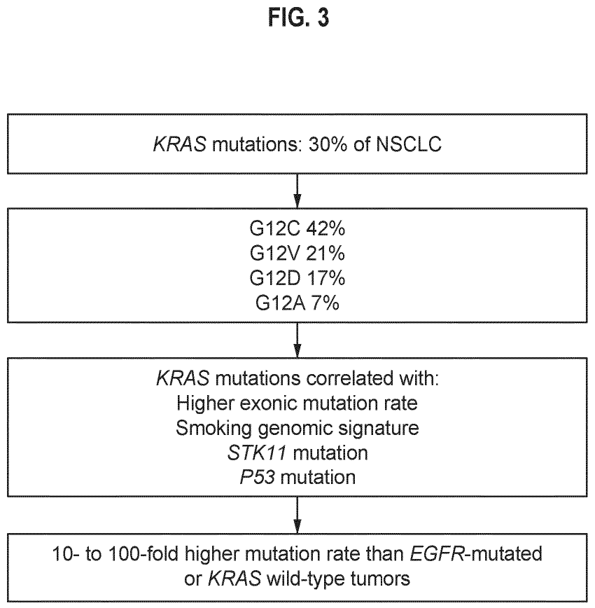

KRAS oncogene is frequently mutated in human tumors and activating mutations in KRAS occur in 20-30% of non-small cell lung cancer (NSCLC) (FIG. 3). KRAS mutations occur mainly in codon 12, 13 or 61. The most common types of KRAS mutations are G12C (42%), G12V (21%), and G12D (17%)2. The KRAS G12C is a single point mutation with a glycine-to-cysteine substitution at codon 12. This substitution favors the activated state of KRAS, amplifying signaling pathways that lead to oncogenesis. KRAS G12C is one of the most common driver mutations in NSCLC and there is a high unmet need, as well as poor outcomes associated in the second-line treatment of KRAS G12C driven NSCLC. In the U.S., about 13% of patients with NSCLC harbor the KRAS G12C mutation, and each year approximately 25,000 new patients in the U.S. are diagnosed with KRAS G12C-mutated NSCLC.

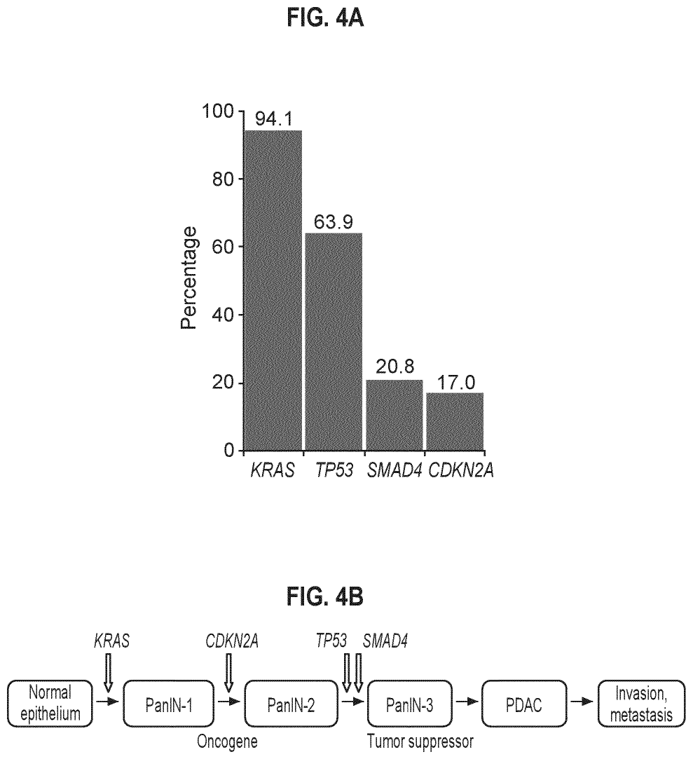

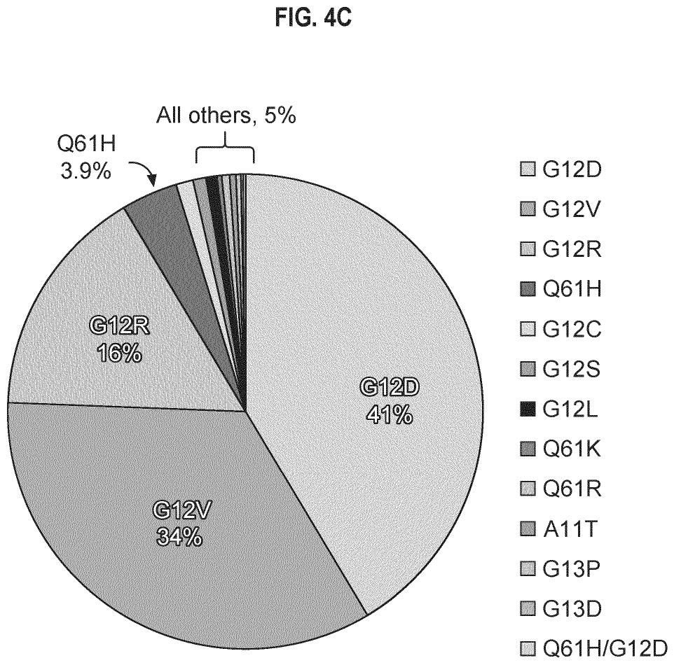

Potential for pan-KRAS treatment would be favorable rather than targeting a single KRAS mutation. The patient population for KRAS mutated NSCLC would be greater since up to 30% of all NSCLC patients possess a KRAS mutation, compared to 13% for G12C single point mutation target. Targeting a pan-KRAS mutated NSCLC treatment could clinically improve the outcomes for many patients. KRAS is mutationally active in 94% of pancreatic ductal adenocarcinoma (PDAC) (FIGS. 4A-4C). The most common mutations are G12D (41%), G12V (34%), and G12R (16%). The G12C mutation is rare in PDAC, with only 1% of all KRAS mutations.

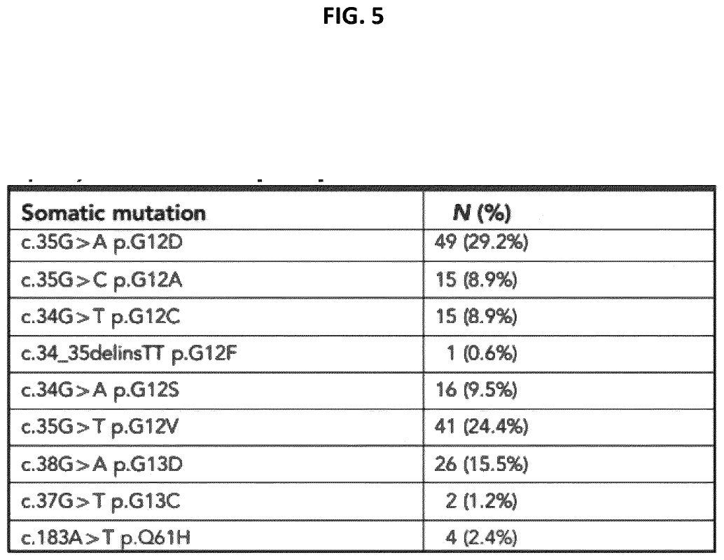

KRAS mutations were detected in 42.9% of 392 evaluated subjects having colorectal cancer (FIG. 5). Within the patients with a codon 12 mutation (136 patients), the most frequent types were G12D (36.0%) and G12V (30.1%). Of the 28 patients with a codon 13 mutation, G13D was the most common point mutation (92.9%). The G12C mutation had a frequency rate of 1.2% (2 out of 392 patients).

### Example 4: Synthetic Lethality in Dual-Targeting of CDK1 and KRAS

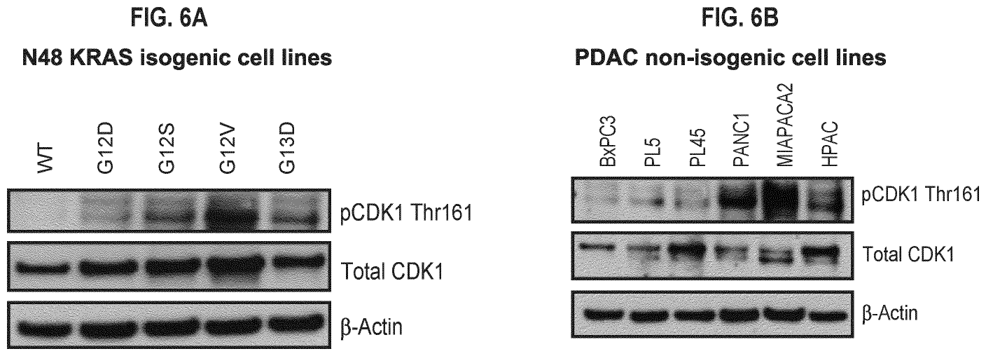

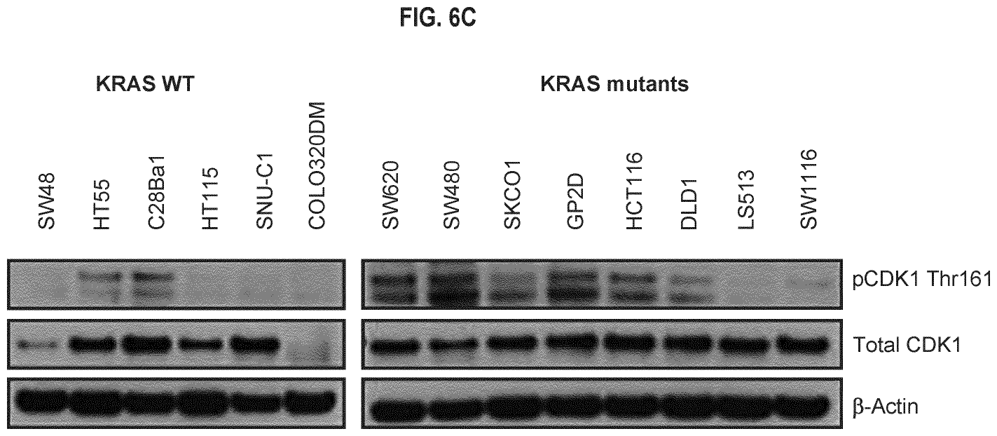

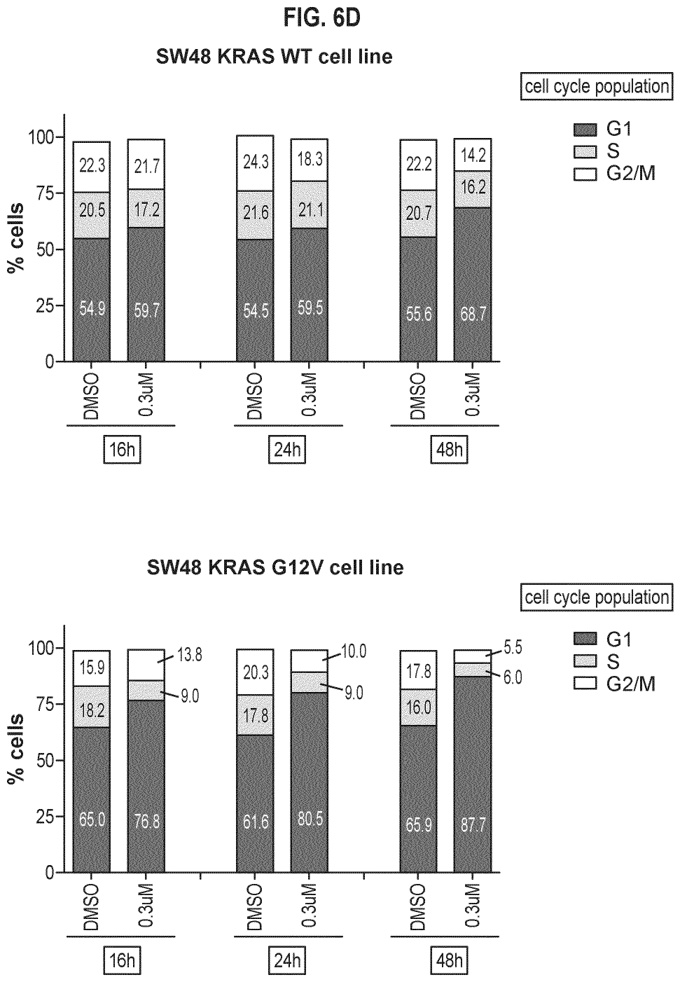

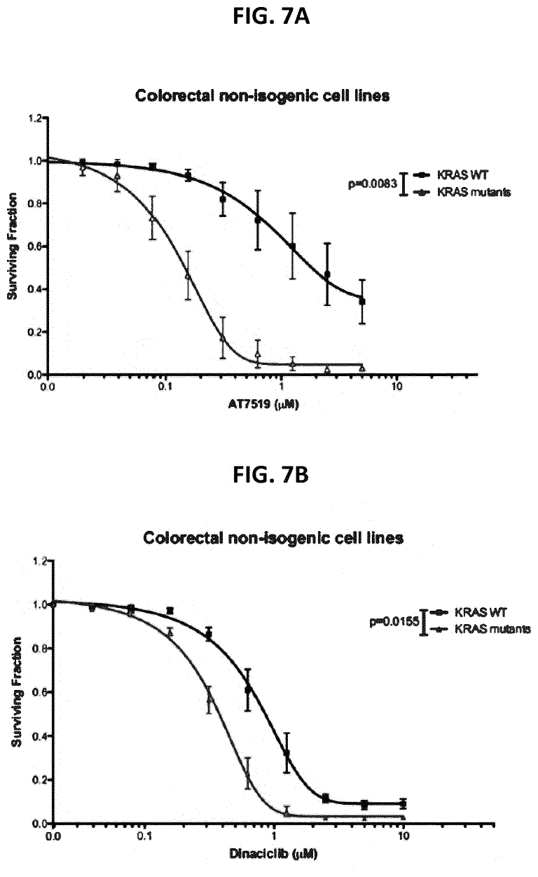

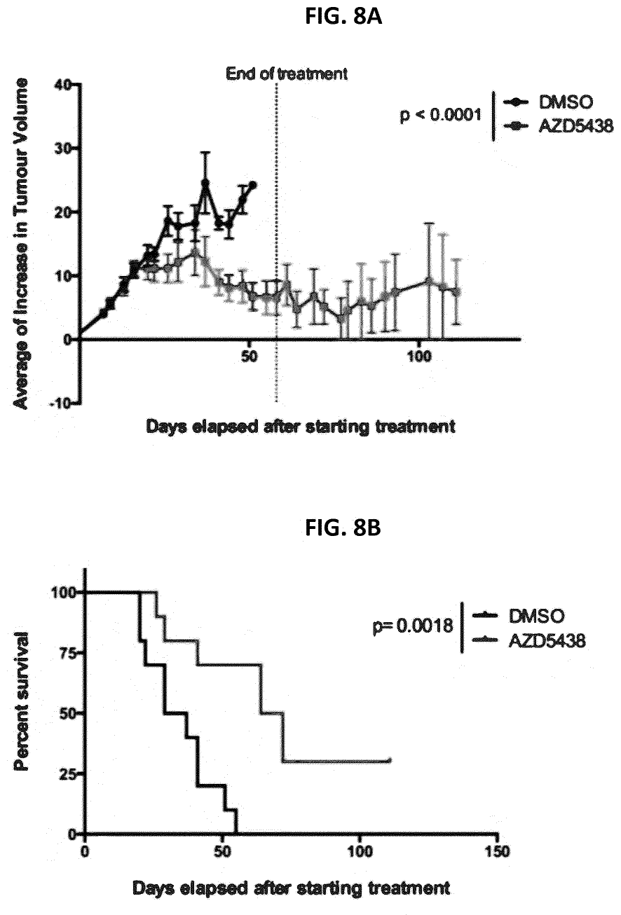

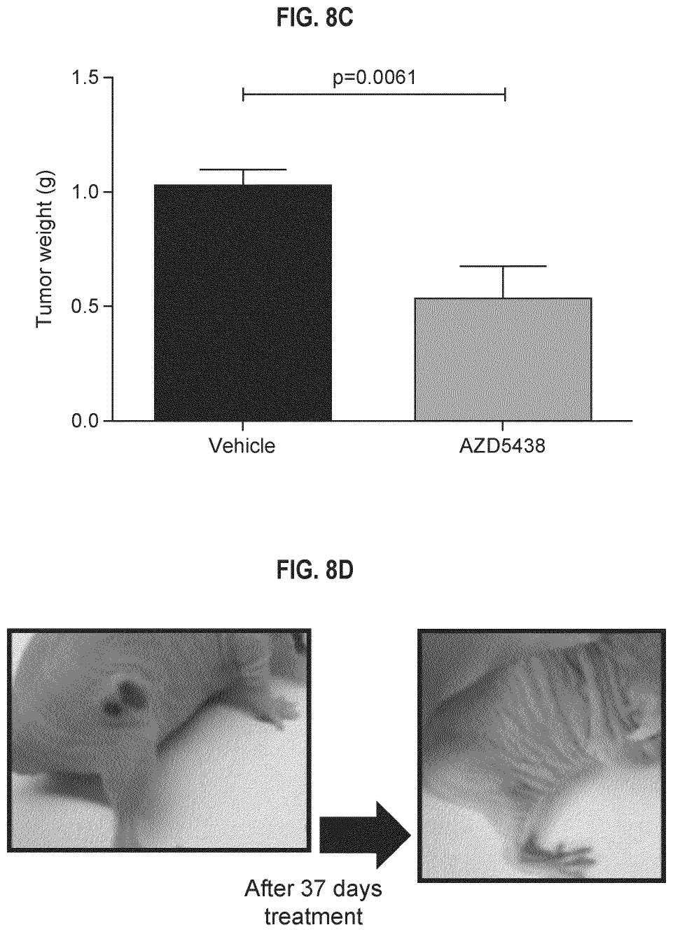

The cyclin-dependent kinase, CDK1, is a synthetic lethal target for KRAS mutant tumors. KRAS mutant cells show increased activity of CDK1 (FIGS. 6A-6D). The inhibition of CDK1 causes a reduction in the S-phase of the cell cycle progression decreasing cell proliferation in KRAS mutant cells. This decreased cell cycle activity is observed by the modulation of Rb, a master control of the G1/S checkpoint. Through a series of experiments, including using parallel siRNA screens in KRAS mutant and wild type colorectal isogenic tumor cells and subsequently validated in a genetically diverse panel of 26 colorectal and pancreatic tumor cell models, the KRAS/CDK1 interaction has a synthetic lethal effect on cancer cells. Cell lines harboring KRAS mutations show increased sensitivity to CDK inhibitors AT519 in vitro (FIGS. 7A-7B) and AZD5438 in vivo (FIGS. 8A-8D).

### Example 5: Milciclib Treatment on KRAS Mutated Cancer in a Mouse Model of Human Lung Adenocarcinomas

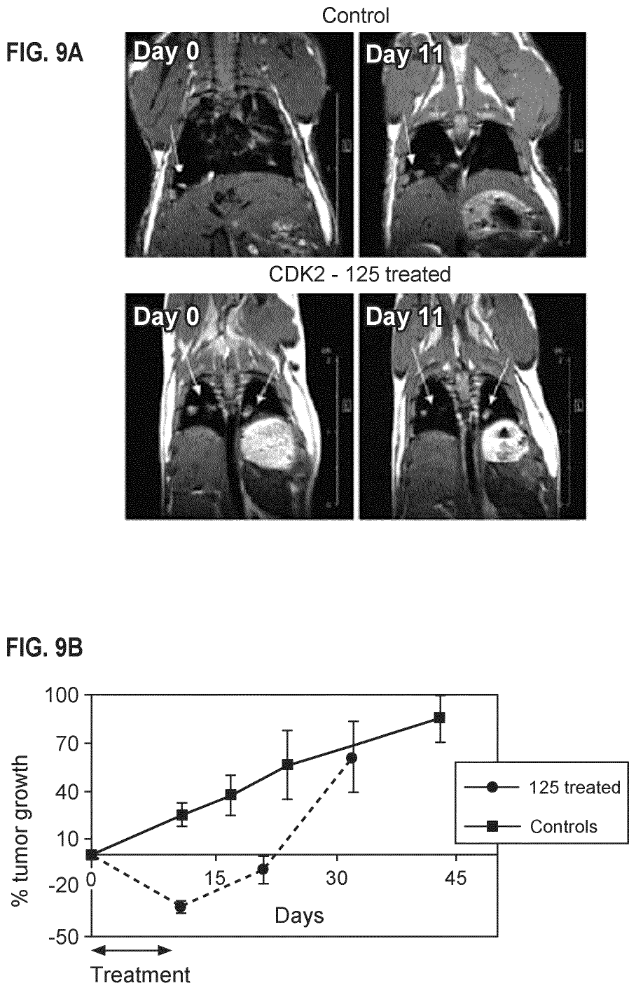

Milciclib was tested in a transgenic mouse model KRAS G12D LA2 that develops pulmonary cancerous lesions reminiscent of human lung adenocarcinomas. Milciclib was tested to follow longitudinal disease progression and evaluate therapeutic efficacy by using magnetic resonance imaging (MRI) and positron emission tomography (PET). Milciclib induced a significant tumor growth inhibition at the end of treatment, as measured by MRI (FIG. 9A) and reduction in tumor growth (FIG. 9B). The MRI methodology used in this study allow longitudinal monitoring of tumor development and response in a single animal, and furthermore, allows for more clinically relevant study design and increases its statistical relevance. Based on the data from this study, milciclib showed significant efficacy in the KRAS G12D LA2 model of NSCLC and could represent a valid alternative to the current treatment of KRAS mutated forms of NSCLC.

### Example 6: Co-Administration of Milciclib and DNA Damaging Agents

For the treatment of cancers with KRAS mutations, a combination treatment of milciclib in addition to a DNA damaging agent would be favorable. These agents induce apoptosis to subsequently eliminate cancer cells from the body. The activity of milciclib has been tested in combination with the following DNA damaging agents: gemcitabine, 5FU, irinotecan and topotecan for pancreatic carcinoma, colon carcinoma, colon carcinoma, and small cell lung carcinoma, respectively. After screening milciclib for in vivo activity in combination with various anticancer agents in xenograft models, the activity of combination treatment was superior to that of each drug alone. It can be safely combined with other anticancer drugs to improve their efficacy.

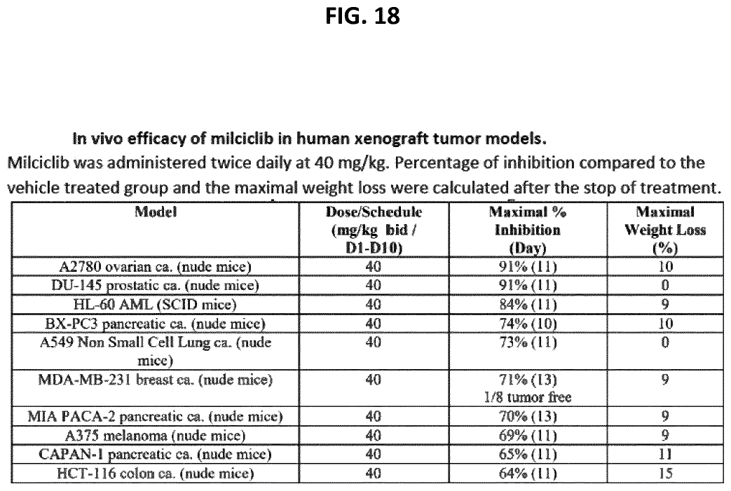

The efficacy of milciclib was tested in several human xenografts implanted in the hind flank region of athymic mice (FIG. 18). Models in nude mice included A2780 ovarian cancer, DU-145 prostatic cancer, BX-PC3 pancreatic cancer, A549 non-small cell lung cancer, MDA-MB-231 breast cancer, MIA-PACA-2 pancreatic cancer, A375 melanoma, CAPAN-1 pancreatic cancer, and HCT-116 colon cancer. The MIA-PACA-2 and CAPAN-1 pancreatic cancer cell lines both express KRAS mutants. The MIA-PACA-2 and CAPAN-1 cell lines tested with milciclib harbor the KRAS mutants, G12C and G12V, respectively. While only 1% of PDAC have the G12C KRAS mutant, 34% express the G12V KRAS mutant. The maximal inhibition of the MIA-PACA-2 xenograft tumor model was 70% and CAPAN-1 showed 65% maximal inhibition. The HCT-116 colon cancer cell line expresses the G13D KRAS mutant, making up of 15.5% of CRC KRAS mutants Following implantation, tumors were allowed to establish to a size of 100 mm3 prior to oral administration of milciclib. In all these experiments, a daily bid treatment was administered for a maximum of 10 days. The compound was effective (maximal TGI from 64% to 91%) and well tolerated (body weight loss from 0% to 15%) in all tested tumor models.

### Co-Administration of Milciclib and DNA Damaging Agents in Colorectal Cancer

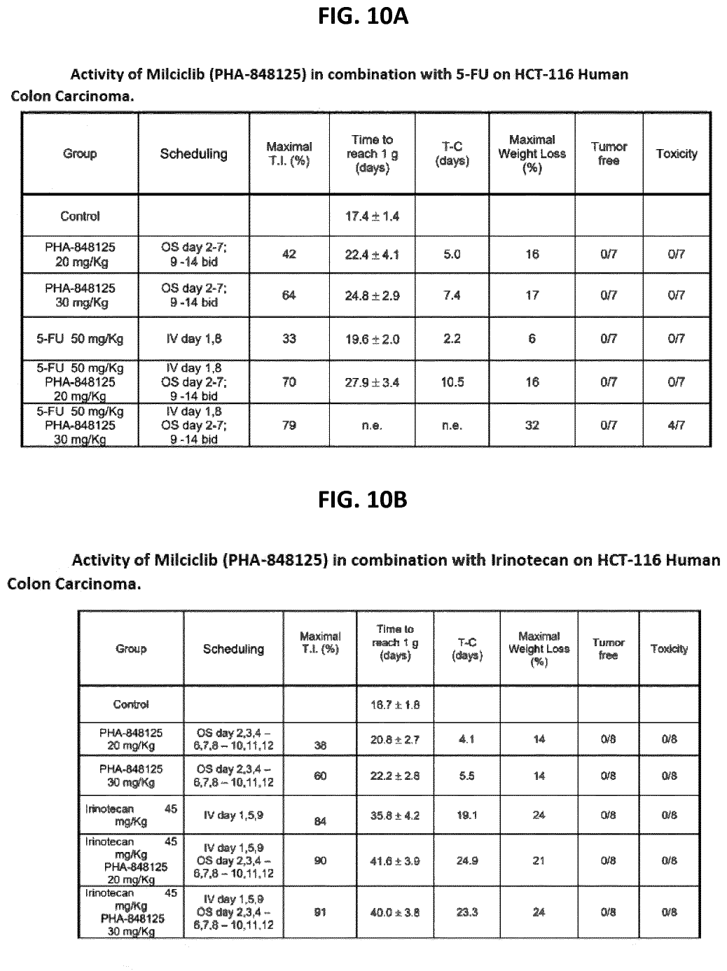

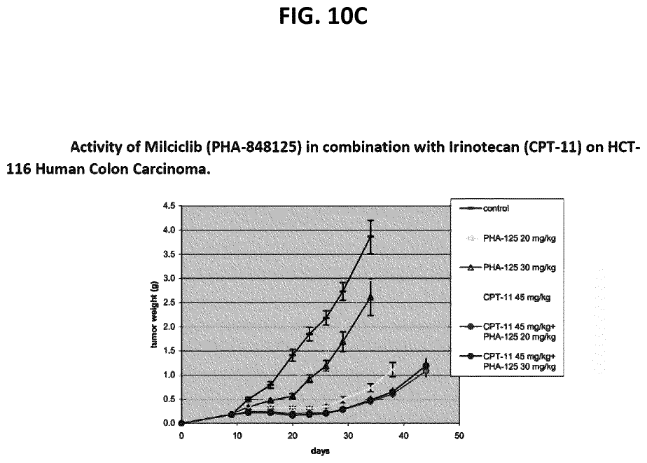

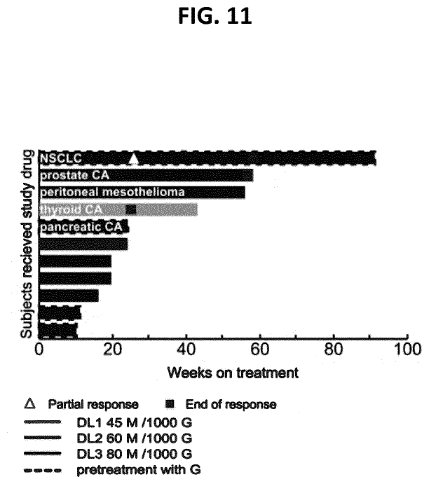

Activity of milciclib has been tested in combination with 5-FU on HCT-116 human colon carcinoma cells (FIG. 10A). The HCT-116 cell line expresses the KRAS G13D mutant. Milciclib was tested orally at the doses of 20 and 30 mg/kg twice a day for 12 days. The obtained TGI values were 42% and 64%, respectively. 5-FU was injected IV at the dose of 50 mg/kg with a q7dx2 schedule and the TGI was 33%. No animal died after these. When milciclib at 20 and 30 mg/kg was combined with 5-FU the TGI was 70% and 79%, respectively. The combination of milciclib at 40 mg/kg and 5-FU at 50 mg/kg dose caused the death of 4 of 7 mice. The T-C value for the combinations with 20 mg/kg milciclib was 10.5 days (expected for additivity: 7.2 days) confirming that the combination produced additive effect.

Another DNA damaging agent, irinotecan, is a topoisomerase I inhibitor (FIG. 10B and FIG. 10C). Topoisomerase enzymes control the manipulation of the structure of DNA necessary for replication. Milciclib was screened for in vivo activity in combination of irinotecan on HCT-116 human colon carcinoma xenograft model. Milciclib was tested orally at the doses of 20 and 30 mg/Kg twice a day for 9 days. The obtained TGI values were respectively of 38 and 60%. Irinotecan was injected IV at the dose of 45 mg/kg with a q4dx3 schedule and the TGI was 84%. No animal died after these. When milciclib 20 and 30 mg/Kg was combined with irinotecan, the TGI was 90 and 91% respectively. No animal died after these treatments. The T-C value for the combinations with 20 mg/kg milciclib was 24.9 days (expected for additivity: 23.2 days) confirming that the combination produced additive effect.

### CO-Administration of Milciclib and DNA Damaging Agents in Non-Small Cell Lung Ganger

Gemcitabine is one of the most effective treatment DNA damaging agents for patients with NSCLC. The pyrimidine nucleoside antimetabolite is a DNA damaging agent and is ranked 3rd in anticancer prescribed worldwide. It has been approved as a monotherapy or in a combination with other drugs for treatment of a variety of solid cancers, including breast cancer, lymphoma, and pancreatic cancer. Toxicities associated with gemcitabine are well known. Several Phase II studies utilizing gemcitabine as a single agent achieved an objective response rate of 20-25% and median survival of 9 months. However, there is tumor recurrence in almost all patients.

Although various chemotherapeutic agents and treatment regimens improved outcomes for patients with advanced NSCLC, the treatments ultimately fail in most patients because of imminent resistance or intolerable toxicity. One way to overcome the resistance to the current standard treatment of gemcitabine monotherapy is a combination treatment with milciclib, reversing the resistance to gemcitabine. Overexpression of CDKs and other downstream signaling pathways that regulate cell cycles have been frequently found to be associated with the development of resistance towards chemotherapies. Inhibition of CDKs is an attractive target for development of small molecule drugs for cancer treatment. As milciclib is a multiple CDK and TrkA inhibitor, the combination treatment of milciclib and gemcitabine can improve efficacy by reversing gemcitabine-resistance and providing enhanced inhibition of cancer, improving patient outcomes.

In a Phase I dose-escalation study, 16 patients were treated with milciclib at 3 dose levels with a fixed dose of gemcitabine. Patients in this study were resistant to standard therapy or for whom no standard therapy exists. Milciclib was administered orally once daily for 7 days on/7 days off in a 4-week cycle, and gemcitabine was administered intravenously on days 1, 8, and 15 in a 4-week cycle. Overall, the combination treatment was well tolerated with manageable toxicities and showed encouraging clinical benefit in ˜36% patients, including gemcitabine refractory patients.

Among the 14 evaluable patients, one NSCLC patient showed a partial response and 4 patients (one each with thyroid, prostatic, pancreatic carcinoma, and peritoneal mesothelioma) showed long-term disease stabilization of >6 to 14 months. In the NSCLC patient with a partial response, the patient was pretreated with gemcitabine as a single agent to which he first responded and later progressed, becoming refractory to gemcitabine. With combination treatment of milciclib and gemcitabine, the patient had a partial response and 6 months of disease stabilization, confirming that the combination treatment of milciclib and gemcitabine does reverse gemcitabine-resistance. The patient with significant disease stabilization of 14.3 months was pretreated with gemcitabine and capecitabine for prostate cancer, showing again that adding milciclib to gemcitabine reverses resistance. The encouraging data from this dose escalation Phase I study shows the combining milciclib and gemcitabine for treatment of refractory solid tumors, most notably NSCLC. The encouraging data from this dose-escalation Phase I study shows the potential improved treatment outcome by combining milciclib and gemcitabine for treatment of refractory solid tumors, most notably NSCLC.

### Co-Administration of Milciclib and DNA Damaging Agents in Pancreatic Cancer

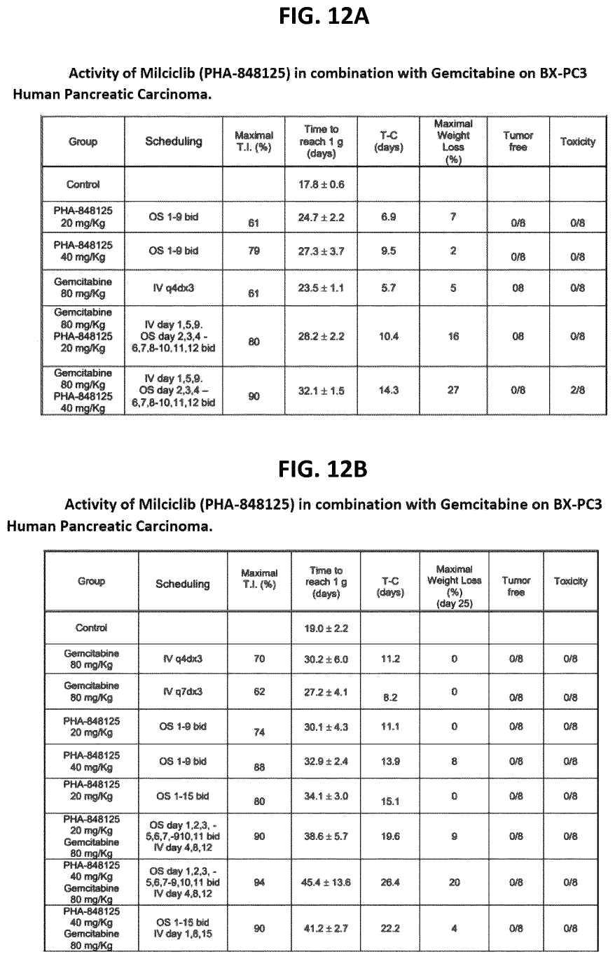

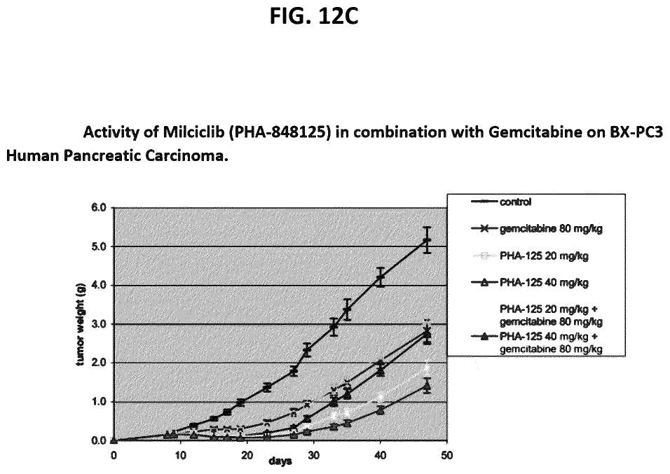

A combination therapy of milciclib and the DNA damaging agent, gemcitabine, was treated on BX-PC3 human pancreatic carcinoma (FIG. 12A). Milciclib was tested orally at the doses of 20 and 40 mg/kg twice a day for 9 consecutive days. The obtained TGI values were respectively of 61% and 79%. Gemcitabine was injected IV at the dose of 80 mg/kg with a TGI of 61%. No animal died after these treatments. When milciclib at 20 and 40 mg/kg was combined with gemcitabine 80 mg/kg, the TGI values were 80% and 90%, respectively. The combination between milciclib 40 mg/kg and gemcitabine 80 mg/kg dose caused the death of 2 of 8 mice. The T-C value for the safe combination of gemcitabine and 20 mg/kg milciclib was 10.4 days (expected for additivity: 12.6 days).

A second experiment was conducted of milciclib in combination with gemcitabine on BX-PC3 human pancreatic carcinoma (FIG. 12B and FIG. 12C). Milciclib was tested orally at the doses of 20 and 40 mg/kg twice a day for 9 consecutive and at 20 mg/kg dose twice for 15 days. The obtained TGI values were 74%, 88% and 80%, respectively. Gemcitabine was injected IV at the dose of 80 mg/kg with a q4dx3 and q7dx3 schedule, and the TGI values were 70% and 62%, respectively. No animal died after these treatments. When milciclib at 20 and 40 mg/kg was combined with gemcitabine 80 mg/kg q4dx3, the obtained TGI were respectively of 90% and 94%, respectively. The 20 mg/kg dose for 15 days of milciclib combined with gemcitabine 80 mg/kg q7dx3 gave a TGI of 90%. No animal died after these treatments. The T-C value for the combination of gemcitabine and 40 mg/kg milciclib was 26.4 days (expected for additivity: 25.1 days) confirming that the combination produced additive effect.

### Co-Administration of Milciclib and DNA Damaging Agents in Small Cell Lung Cancer

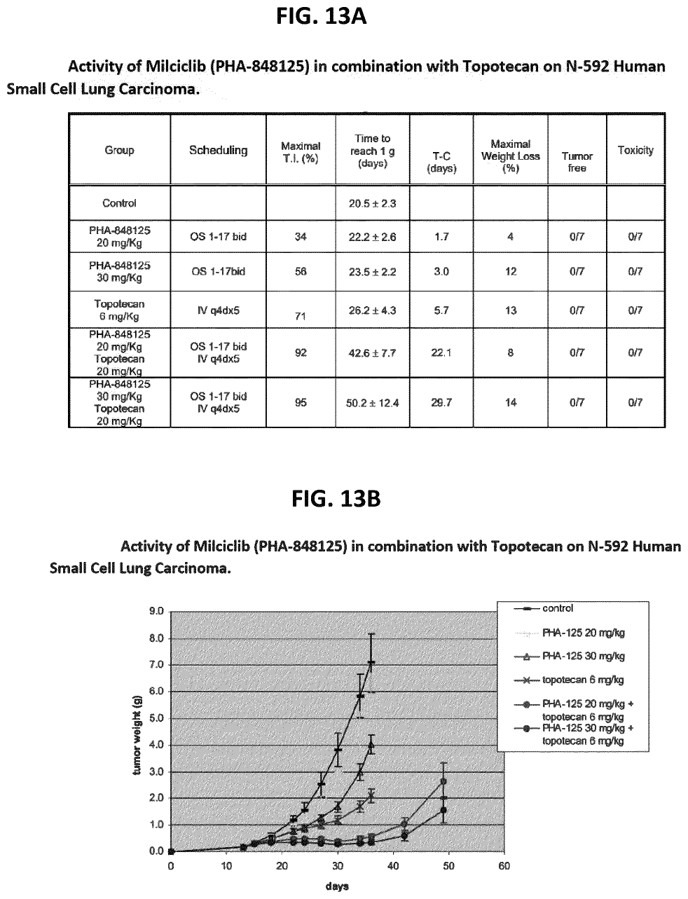

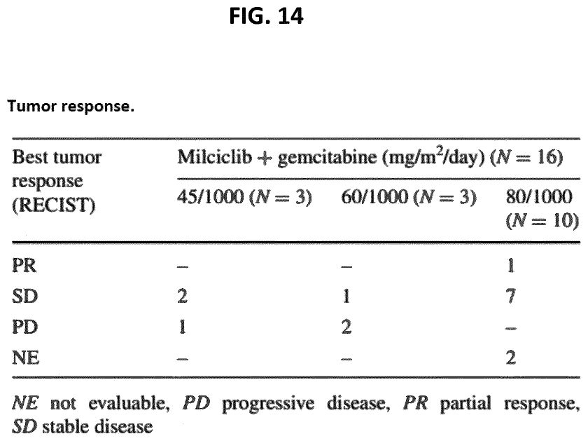

The combination of topotecan and milciclib was screened on N-592 human small cell carcinoma (FIG. 13A and FIG. 13B). Milciclib was tested orally at the doses of 20 and 30 mg/kg twice a day for 17 consecutive days. The obtained TGI values were respectively of 34 and 56% topotecan was injected IV at the dose of 6 mg/kg with a q4dx5 schedule and the TGI was 71%. No animal died after these treatments. When milciclib 20 and 30 mg/kg was combined with topotecan the TGI was 92 and 95% respectively, without any sign of toxicity. The T-C values were 22.1 and 29.7 days for the combinations with 20 mg/kg and 30 mg/kg milciclib respectively (expected for additivity: 7.4 and 8.7 days, respectively) confirming that the combination produced more than additive effect.

### Co-Administration of Milciclib and DNA Damaging Agents in Breast Cancer

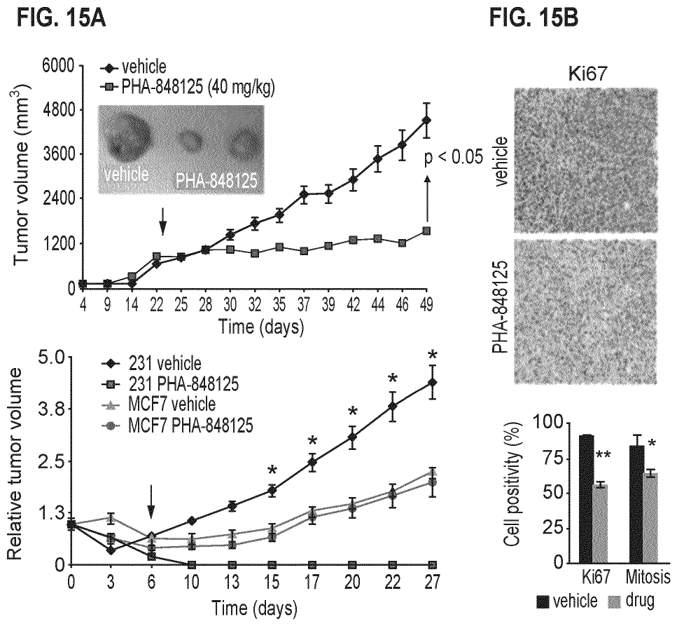

The in vivo efficacy of milciclib using the MDA-MBA-231 xenograft mouse model. Milciclib arrests tumor growth with a consistent reduction in the proliferation marker Ki67 and the number of mitotic nuclei (FIG. 15A and FIG. 15B). He also proves milciclib specifically arrested growth in triple negative breast cancer (TNBC) tumors without affecting the ER-positive tumors. The in vivo efficacy of milciclib after cyclic treatments was tested in MDA-MB-231 xenotransplant mice to prove after multiple treatments, tumors continued to respond to milciclib and appeared to be well tolerated.

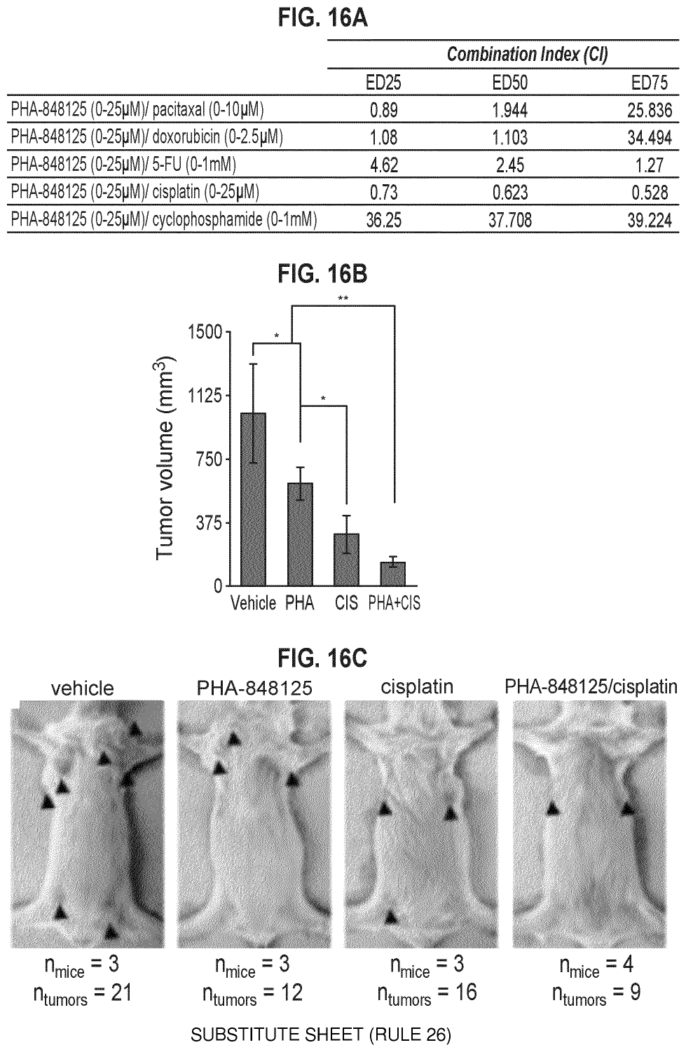

The use of milciclib in combination with five chemotherapeutic drugs already in use was tested for the treatment of TNBC including paclitaxel, doxorubicin, 5-FU, cisplatin, and cyclophosphamide. Potential synergistic effects with milciclib by exposing MDA-MB-231 cells to serial dilutions of milciclib and each DNA damaging agent, alone or in combination, in clinically relevant concentrations (FIG. 16A). Evaluation of the in vivo efficacy for the combination in MMTV-PyMT transgenic mice showed a 2.3-fold reduction in the number of tumors after treatment in comparison to vehicle treated mice (FIG. 16B and FIG. 16C). Tumor proliferation was reduced by nearly 5 times for the combination treatment of milciclib and cisplatin compared to the vehicle treated group, assessed by Ki67 staining. Data shows that milciclib acts synergistically with cisplatin to enhance cisplatin-induced apoptosis in TNBC cells.

### Co-Administration of Milciclib and DNA Damaging Agents in Ovarian Cancer

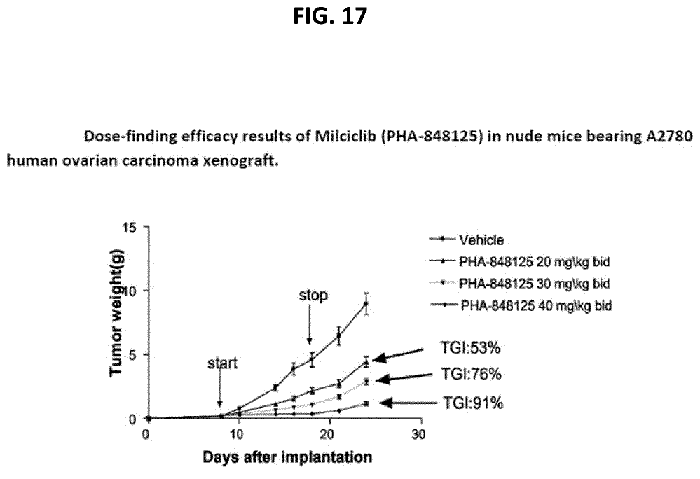

Pathogenic KRAS mutations were present in 606 patients of 7,325 epithelial ovarian cancers from the Caris Database, about 8.3% of the total. The frequency of KRAS mutation by subtype occurred in the following: 36.4% G12D, 35.8% G12V, and 8.4% G12C. A dose-dependent inhibition of tumor growth was measured in mice harboring A2780 human ovarian carcinoma xenografts administered with daily oral administration of milciclib. Significant tumor growth inhibition (TGI) was seen at the doses of 30 and 40 mg/kg bid (76% and 91%, respectively). At the lowest dose of 20 mg/kg bid, TGI was 53% (FIG. 17).

### Example 7: Co-Administration of Milciclib and PARP Inhibitors

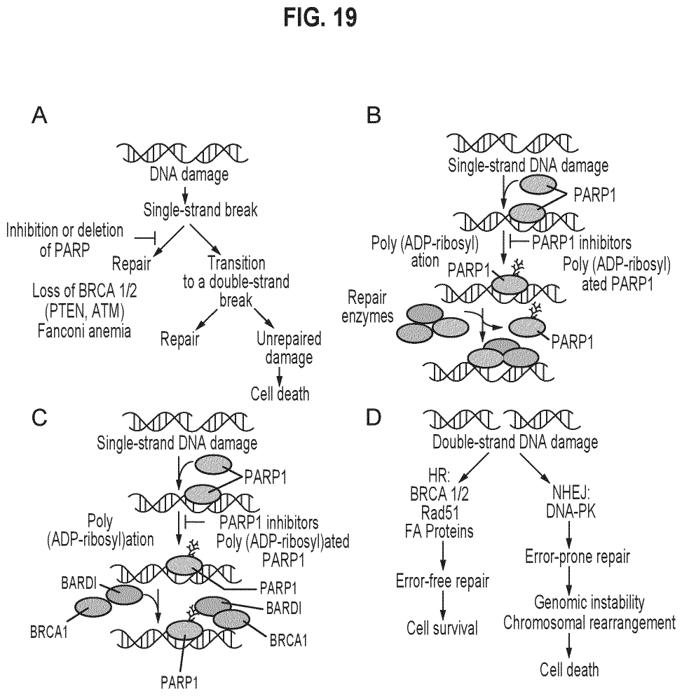

PARP inhibitors can be another cytotoxic agent that disrupts the DNA damage response (DDR) within tumor cells in the combination therapy with milciclib. Poly adenosine diphosphate-ribose polymerase (PARP) is an enzyme that helps repair DNA damage, specifically the base excision repair (BER) in single strand breaks, in cells (FIG. 19). PARP inhibitors selectively bind to PARP to prevent DNA repair and are an approved treatment for BRCA1/2 mutant pancreatic, breast, ovarian and prostate cancer patients. Four current approved PARP inhibitors are olaparib (Lynparza), niraparib (Zejula), rucaparib (Rubraca), and talazoparib (Talzenna).

We claim:

1. A method of treating a cancer in a subject in need thereof, the method comprising:
a. identifying a subject with a having a KRAS mutant tumor; and
b. administering milciclib to the subject.

2. The method of claim 1, further comprising administering a DNA damaging agent to the subject.

3. The method of claim 2, wherein the administration of milciclib and the DNA damaging agent is concurrent or sequential.

4. The method of any one of the preceding claims, wherein the DNA damaging agent is a poly adenosine diphosphate-ribose polymerase (PARP) inhibitor, a Topoisomerase I inhibitor, a Topoisomerase II inhibitor, an alkylating agent, an alkylating agent-steroid conjugate, an epoxide, a platin drug, an anthracenedione, an antimetabolite, an antifolate, a nucleic acid analog, a ribonucleic acid analog, a ribozyme, radiation, a vinca alkaloid, FOLFIRI, or a taxane.

5. The method of claim 4 wherein the platin is cisplatin, oxaliplatin or carboplatin.

6. The method of claim 4, wherein the antimetabolite is a gemcitabine, or a 5-fluorouracil.

7. The method of claim 4, wherein the Topoisomerase I inhibitor is topotecan or irinotecan.

8. The method of claim 4, wherein the Topoisomerase II inhibitor is anthracycline.

9. The method of claim 4, wherein the alkylating agent is nitrogen mustard, a nitrourea, alkyl sulfonate a triazine, an aziridine or an ethylenimine.

10. The method of any one of the preceding claims, wherein the KRAS mutant tumor has a one or more mutations anywhere on the KRAS gene.

11. The method of claim 10, wherein the KRAS mutation occurs in codon 12, codon 13, or codon 61 of the KRAS gene.

12. The method of claim 10 wherein the KRAS mutation is at least one of G12D, G12F, G12V, G12R, Q61H, G12C, G12S, G12L, Q61K, Q61R, A11T, G13C, G13P, G13D, and O51H.

13. The method of any one of the preceding claims, wherein the cancer is selected from non-small cell lung cancer, small cell lung carcinoma, colorectal cancer, pancreatic cancer, breast cancer, ovarian cancer, biliary cancer and melanoma.

14. The method of any one of the preceding claims, wherein the subject has failed one or more previous treatment regimens.

15. The method of any one of the preceding claims, wherein the cancer is refractory to one or more prior administered chemotherapies.

16. The method of any one of the preceding claims, wherein the cancer is sensitized to the one or more prior administered therapies following administration of milciclib.

17. The method of any one of the preceding claims, wherein the cancer is gemcitabine-resistant prior to administering milciclib.

18. The method of any one of the preceding claims, wherein the cancer is sensitized to gemcitabine following administration of milciclib.

19. The method of any one of the preceding claims, wherein the subject is a human.

20. The method of any one of the preceding claims, wherein the milciclib is administered as a unit dose, wherein the unit dose is a therapeutically effective amount.

21. The method of claim 20, wherein the unit dose is about 20 mg/kg, about 30 mg/kg, about 40 mg/kg, about 50 mg/kg, about 60 mg/kg, about 70 mg/kg, or about 80 mg/kg.

22. The method of claim 20, wherein the unit dose is 20 mg per day, 25 mg per day, 30 mg per day, 35 mg per day, 40 mg per day, 45 mg per day, 50 mg per day, 55 mg per day, 60 mg per day, 65 mg per day, 70 mg per day, 75 mg per day, 80 mg per day, 85 mg per day, 90 mg per day, 95 mg per day, 100 mg per day, 105 mg per day, 110 mg per day, 115 mg per day, 120 mg per day, 125 mg per day, 130 mg per day, 135 mg per day, 140 mg per day, 145 mg per day, 150 mg per day, 155 mg per day, or 160 mg per day.

23. The method of any one of the preceding claims, wherein the unit dose is administered orally.

24. The method of any one of the preceding claims, wherein the unit dose is administered once a day or twice a day.

25. The method of any one of the preceding claims, wherein the unit dose is administered for about 7 consecutive days, about 9 consecutive days, or about 15 consecutive days.

26. The method of any one of the preceding claims, wherein the unit dose is administered for a cycle of 7 days on followed by 7 days off, wherein the cycle is repeated for 4 weeks.

27. The method of any one of the preceding claims, wherein the unit dose is administered for a cycle of 4 days on followed by 3 days off, wherein the cycle is repeated for 4 weeks.

28. The method of any one of the preceding claims, wherein the therapeutically effective amount of gemcitabine is 1000 mg/m² over 30 minutes once weekly for seven weeks, followed by one week of no administration, wherein the cycle is optionally repeated.

29. The method of any one of the preceding claims, wherein the therapeutically effective amount of milciclib is 50, 75, 100, 125, or 150 mg once daily for four consecutive days, followed by non-administration for 3 consecutive days, wherein the cycle is optionally repeated.

30. The method of any one of the preceding claims, wherein milciclib and the other anticancer drug are administered to the patient simultaneously.

31. The method of any one of the preceding claims, wherein milciclib and the other anticancer drug are administered in a single pharmaceutical formulation that further includes a pharmaceutically acceptable excipient.

32. The method of claim 31, wherein the pharmaceutical formulation is in a controlled release form.

33. The method of any one of the preceding claims, wherein milciclib and the other anticancer drug are each administered in separate pharmaceutical formulations, wherein each formulation further includes a pharmaceutically acceptable excipient.

34. The method of claim 33, wherein one or both of the pharmaceutical formulations is in a controlled release form.

35. The method of any one of the preceding claims, wherein milciclib and the other anticancer drug are administered to the subject sequentially.

36. The method of any one of the preceding claims, wherein administration of milciclib begins before administration of the other DNA damaging agent to the subject.

37. The method of any one of the preceding claims, wherein administration of milciclib begins after administration of the other anticancer to the subject.

38. The method of any one of the preceding claims, wherein milciclib is administered in a single pharmaceutical formulation that further includes a pharmaceutically acceptable excipient.

39. The method of any one of the preceding claims, wherein the pharmaceutical formulation is formulated for oral administration.

40. The method of claim 39, wherein the pharmaceutical formulation is in the form of a tablet, pill, or capsule.

41. The method of any one of the preceding claims, wherein milciclib and the DNA damaging agent are administered in temporal proximity.

42. A pharmaceutical composition comprising milciclib or a pharmaceutically acceptable salt, isomer, or tautomer thereof, and another anticancer drug.

43. A kit comprising:
(a) a pharmaceutical composition comprising milciclib, or a pharmaceutically acceptable salt thereof; and
(b) a pharmaceutical composition comprising a poly adenosine diphosphate-ribose polymerase (PARP) inhibitor, a Topoisomerase I inhibitor, a Topoisomerase II inhibitor, an alkylating agent, an alkylating agent-steroid conjugate, an epoxide, a platin, an anthracenedione, an antimetabolite, an antifolate, a nucleic acid analog, a ribonucleic acid analog, a ribozyme, radiation, a vinca alkaloid, FOLFIRI, or a taxane, sorafenib, lenvatinib, regorafenib, sunitinib, nivolumab, gemcitabine, palbociclib, afatinib, alectinib, axitinib, bortezomib, bosutinib, cabozantinib, carfilzomib, ceritinib, cobimetinib, crizotinib, dabrafenib, erlotinib, gefitinib, ibrutinib, idelalisib, imatinib, ixazomib, lapatinib, nilotinib, nintedanib, niraparib, osimertinib, pazopanib, pegaptanib, ponatinib, rucaparib, ruxolitinib, sonidegib, tofacitinib, trametinib, vandetanib, vemurafenib, vismodegibor, or a pharmaceutically acceptable salt thereof, and
(c) instructions for the use thereof in the treatment and/or prevention of cancer.
